(19) United States
(12) Patent Application Publication
(10) Pub. No.: US 2018/0271969 A1
(43) Pub. Date: Sep. 27, 2018

(54) STREPTOCOCCUS SUIS POLYSACCHARIDE-PROTEIN CONJUGATE COMPOSITION

(71) Applicants: Boehringer Ingelheim Vetmedica, Inc., Saint Joseph, MO (US); Université de Montréal, Montréal (CA); Université du Québec à Montréal, Montréal (CA)

(72) Inventors: Marcelo GOTTSCHALK, Saint-Hyacinthe (CA); Guillaume GOYETTE-DESJARDINS, Saint-Hyacinthe (CA); Jennifer Anne Kempker, Lee's Summit, MO (US); Axel NEUBAUER, Savannah, MO (US); René ROY, Terrebonne (CA); Mariela SEGURA, Saint-Hyacinthe (CA); Tze Chieh SHIAO, Montréal (CA)

(73) Assignees: Boehringer Ingelheim Vetmedica, Inc., Saint Joseph, MO (US); Université de Montréal, Montreal (CA); Université du Québec à Montréal, Montreal (CA)

(21) Appl. No.: 15/760,891

(22) PCT Filed: Oct. 6, 2016
(86) PCT No.: PCT/US2016/055658

§ 371 Date: Mar. 16, 2018

Related U.S. Application Data
(60) Provisional application No. 62/238,516, filed on Oct. 7, 2015.

(51) Int. Cl. A61K 39/09

(52) U.S. Cl. CPC A61K 39/092; A61K 2039/55544

(57) ABSTRACT

The present invention relates to an immunogenic composition comprising polysaccharide-protein conjugates wherein each conjugate contains a capsular polysaccharide prepared from Streptococcus suis serotypes 1, 2, 7 and/or 9 or any other serotype conjugated to a carrier protein. The immunogenic composition is useful for the protection of disease in an animal subject.

### BACKGROUND OF THE INVENTION

### A. Field of the Invention

The present invention relates to an immunogenic composition comprising polysaccharide-protein conjugates. In one embodiment the conjugate contains a capsular polysaccharide, for example, prepared from Streptococcus suis serotypes, including but not limited to serotypes 1, 2, 7 and/or 9, conjugated to a carrier protein. The immunogenic composition is useful for the protection of S. suis associated disease in swine.

### B. Description of the Related Art

Streptococcus suis is a Gram-positive encapsulated bacterium and one of the most important bacterial pathogens in the porcine industry, resulting in important economic losses (Gottschalk. Diseases of swine. 10th ed.; 2012. p. 841-55). Initial reports of infection by this pathogen were published in the Netherlands (1951) and in England (1954), followed by characterization of septicemic pigs isolates by de Moor between 1956 and 1963 as new Lancefield groups (Field et al. Vet Rec. 1954; 66:453-5; Jansen and Dorssen. Tijdschr Diergeneeskd. 1951; 76:815-32; de Moor C E. Antonie Van Leeuwenhoek. 1963; 29:272-80). In the following years, the species was named Streptococcus suis (Elliott S D. J Hyg (Lond). 1966; 64:205-12; Windsor and Elliott J Hyg (Lond). 1975; 75:69-78).

To date, over 30 S. suis serotypes have been described based on the capsular polysaccharide (CPS) antigenic diversity, and S. suis serotype 2 is considered the most virulent and most frequently isolated from clinical samples and associated with disease in pigs (Goyette-Desjardins et al. Emerg Microbes. Infect 2014; 3:e45). S. suis, mainly serotype 2, is also an important emerging zoonotic agent for persons in close contact with pigs or pig-derived products Id.

The natural habitat of S. suis is the upper respiratory tract of pigs, more particularly the tonsils and nasal cavities, as well as the genital and digestive tracts (Higgins and Gottschalk Diseases of swine. 2006. p. 769-83). Transmission of S. suis among animals is considered to be mainly through the respiratory route. Id. Of the various manifestations of the disease, septicemia and meningitis are by far the most striking features, but endocarditis, pneumonia, arthritis, and other clinical outcomes can also be observed (Sanford and Tilker J Am Vet Med Assoc. 1982; 181:673-6). Nevertheless, in peracute cases of infection, pigs are often found dead with no premonitory signs of disease. Id. Although the incidence of disease in swine varies over time and is generally less than 5%, mortality rates can reach 20% in the absence of treatment (Cloutier et al. Vet Microbiol. 2003; 97:135-51). Affected animals are generally between 5 and 10 weeks of age, but infections have also been reported from newborn piglets to 32 week-old pigs (Higgins and Gottschalk, 2006).

The thick surface-associated S. suis CPS confers the bacteria protection against the immune system, notably by resisting phagocytosis (Segura M. Can J Microbiol 2012; 58:249-60). As with most extracellular encapsulated bacteria, protection against S. suis is therefore likely mediated by opsonizing antibodies, which induce bacterial clearance by opsonophagocytosis. Research has been ongoing for years in the hope of developing an efficient vaccine to protect against S. suis disease. Yet, no such vaccine is available. Commercial or autogenous killed whole-cell vaccines (bacterins) are used in the field with poor results (Gottschalk. Diseases of swine. 2012. p. 841-55; Lapointe et al. Can J Vet Res. 2002; 66:8-14; Baums et al. Clin Vaccine Immunol. 2010; 17:1589-97; Wisselink H J, et al. Vet Microbiol. 2002; 84:155-68). Other strategies have been experimentally tested such as live strains and sub-unit vaccines. The use of live avirulent strains gave inconsistent results and may present some safety concerns (zoonosis) (Baums et al., 2010; Busque et al. Can J Vet Res. 1997; 275-9; Fittipaldi et al. Vaccine. 2007; 25:3524-35). After several years of research, there is still no proven and commercially available protein-based subunit vaccine using well characterized virulence factors and/or protective antigens (Fittipaldi et al. Vaccine. 2007; 25:3524-35). Being a pathogen with a multifactorial virulence mechanism and presenting a relatively high phenotypic heterogeneity, these findings are, to a certain extent, expected (Goyette-Desjardins et al., 2014; Fittipaldi et al. Future Microbiol. 2012; 7:259-79). Additionally, while bacterins have been shown to be effective when combined with potent adjuvants, these combinations have been shown to be highly reactive. The resulting side-effects make such a combination commercially undesirable.

It has been reported that anti-CPS antibodies have a high protective potential in the fight against infection by S. suis, yet this bacterial component is poorly immunogenic (Calzas et al. Infect Immun. 2015; 83:441-53; Charland et al. Microbiology. 1997; 143:3607-14).

Polysaccharides/carbohydrates, unlike proteins and peptides, are generally recognized as T cell-independent antigens, explaining their innate inability to stimulate helper T cells via MHC class-II signaling, resulting in low immune cell proliferation, no antibody class switching or affinity/specificity maturation, and more importantly, lack of immunological memory (Roy and Shiao. Chimia. 2011; 65:24-9). Yet, some purified bacterial CPSs, such as those from S. pneumoniae (PNEUMOVAX®—23 valent) and from Group B Streptococcus (GBS) serotype III can induce not only IgM but also IgG antibody responses in mice and in adults (Heath P T. Expert Rev Vaccines. 2011; 10:685-94; Baker et al. N Engl J Med. 1988; 319:1180-5; Kasper et al. J Clin Invest. 1996; 98:2308-14; Moens et al. Infect Immun. 2009; 77:1976-80; Schütz et al. J Clin Immunol. 2013; 33:288-96). Other CPSs need to be properly conjugated to protein carriers serving as T cell-dependent epitopes (a composition named as glycoconjugate), rendering these bacterial CPSs potent vaccine antigens. Glycoconjugate vaccines have demonstrated success in the fight against encapsulated bacteria in human medicine, such as vaccines against Haemophilus influenzae (HIBERIX®), Neisseria meningitidis (MENACWY®), and Streptococcus pneumoniae (PCV13®) (See U.S. Pat. No. 7,709,001) Id. Despite the popular use of glycoconjugate vaccines in human medicine, this strategy has been poorly developed for veterinary practice. S. suis serotype 2 CPS alone is unable to induce any significant antibody response, even when adjuvanted with TITERMAX® Gold or STIMUNE® or when combined with the TLR-ligand CpG (unpublished results). Furthermore, previous studies using live S. suis serotype 2 infection showed modest IgM and no isotype-switched IgG specific anti-CPS antibody titers in pigs and in mice even after an experimental re-infection (Calzas et al. Infect Immun. 2015; 83:441-53). A precedent exists in the literature where serotype 2 CPS was conjugated to bovine serum albumin with the aim to obtain anti-CPS control sera for in vitro studies (Baums et al. Clin Vaccine Immunol, 2009, 16:200-8). Yet, neither the biochemical characteristics nor the immunogenicity and functional activity of that glycoconjugate were investigated.

What is needed is a highly-protective and efficacious vaccine of reduced reactivity compared to efficacious bacterin vaccines. Currently there are no commercially available effective vaccines against S. suis infection in swine. While an efficacious serotype 2 vaccine would provide substantial benefits, a cross-protective vaccine providing protection against most important serotypes of S. suis would be preferred.

### SUMMARY OF THE INVENTION

The present invention provides immunogenic compositions, vaccines, and related methods that overcome deficiencies in the art. The compositions and methods provide protection of swine from disease caused by Streptococcus suis infection caused by different serotypes, including but not limited to serotypes 1, 2, 7 and/or 9, in particular the clinical signs of S. suis infection including, for example, meningitis, septicemia, endocarditis, arthritis, and septic shock.

The present invention provides monovalent (one serotype) immunogenic compositions, comprising polysaccharide-protein conjugates, together with a physiologically acceptable vehicle, wherein the S. suis capsular polysaccharides (CPS s) are from selected from the group comprising S. suis serotypes 1, 2, 7, and 9, or any other serotype, wherein the CPS is coupled to a protein carrier.

Immunogenic compositions and vaccines of the invention comprise bacterial capsular polysaccharides conjugated to a protein carrier, for example, in one non-limiting embodiment the tetanus toxoid protein.

In yet a further aspect, immunogenic compositions can be multivalent (multiple serotypes) immunogenic compositions, comprising polysaccharide-protein conjugates, together with a physiologically acceptable vehicle, wherein each of the conjugates comprises a capsular polysaccharide from a different serotype of S. suis conjugated to a carrier protein, and the capsular polysaccharides are prepared from 1, 2, 7 and 9, or any other serotype, and any combination thereof.

The present invention also provides monovalent and multivalent conjugated vaccines for S. suis conferring cross-protection against serotypes 2 and/or 1, 7 and 9 or any other serotype.

Those of skill in the art will understand that the compositions used herein may incorporate known injectable, physiologically acceptable sterile solutions. For preparing a ready-to-use solution for parenteral injection or infusion, aqueous isotonic solutions, e.g. saline or plasma protein solutions, are readily available. In addition, the immunogenic and vaccine compositions of the present invention can include veterinary-acceptable carriers, diluents, isotonic agents, stabilizers, or adjuvants.

Methods of the invention include, but are not limited to, a method of provoking an immune response against a S. suis infection in a subject comprising the step of administering to the subject an immunogenic composition comprising one or more bacterial capsular polysaccharides conjugated to a protein carrier as defined herein. Preferably, the immune response is provoked against more than one serotype or strain of S. suis. Compositions of the invention may be used to prevent a S. suis infection. Preferably, such immune response reduces the incidence of or severity of one or more clinical signs associated with or caused by the infection with one or more S. suis serotypes.

Herein, suitable subjects and subjects in need to which compositions of the invention may be administered include swine and herds of swine in need of prophylaxis for S. suis infection.

The invention also provides a method of reducing the incidence of or severity of one or more clinical signs associated with or caused by S. suis infection, comprising the step of administering an immunogenic composition of the invention that comprises one or more polysaccharide-protein conjugates comprising S. suis serotypes 1, 2, 7, and 9, or any other serotype, or combinations thereof, as provided herewith, such that the incidence of or the severity of a clinical sign of the S. suis infection is reduced by at least 10%, preferably at least 20%, even more preferred at least 30%, even more preferred at least 50%, even more preferred at least 70%, most preferred at least 100% relative to a subject that has not received the immunogenic composition as provided herewith. Such clinical signs can include, for example, behavioral changes, lameness, death, meningitis, septicemia, endocarditis, arthritis, and septic shock. And, any of these clinical signs may result from an infection with a S. suis due to infection with serotype 1, 2, 7, and 9 or any other serotype of S. suis.

Methods of making immunogenic compositions of the invention may further comprise admixing the S. suis polysaccharide-protein conjugates with a physiologically-acceptable vehicle such as a pharmaceutically- or veterinary-acceptable carrier, adjuvant, or combination thereof. Those of skill in the art will recognize that the choice of vehicle, adjuvant, or combination will be determined by the delivery route, personal preference, and animal species among others.

The invention also provides kits that comprise an immunogenic composition that comprises one or more S. suis polysaccharide-protein conjugates; a container for packaging the immunogenic composition; a set of printed instructions; and a dispenser capable of administering the immunogenic composition to an animal. The invention also provides kits for vaccinating an animal comprising a set of printed instructions; a dispenser capable of administering the immunogenic composition provided herewith comprising one or more S. suis polysaccharide-protein conjugates to an animal; and wherein at least one of S. suis polysaccharide-protein conjugates effectively immunizes the animal against at least one clinical sign associated with S. suis infection. Kits of the invention may further comprise a veterinary acceptable carrier, adjuvant, or combination thereof.

Those of skill in the art will understand that the compositions used herein may incorporate known injectable, physiologically acceptable sterile solutions. For preparing a ready-to-use solution for parenteral injection or infusion, aqueous isotonic solutions, e.g. saline or plasma protein solutions, are readily available. In addition, the immunogenic and vaccine compositions of the present invention can include pharmaceutical- or veterinary-acceptable carriers, diluents, isotonic agents, stabilizers, or adjuvants.

Methods of the invention may also comprise mixing a composition of the invention with a veterinary acceptable carrier, adjuvant, or combination thereof. Those of skill in the art will recognize that the choice of carrier, adjuvant, or combination will be determined by the delivery route, personal preference, and animal species among others.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1E: The presence of conjugates in the different preparations verified by Gel shift and Western blot experiments. FIG. 1A: Gel shift experiments, Coomassie Blue, and FIG. 1B: Gel shift experiments, Silver staining demonstrating a considerable shift from the purified TT monomer at 150 kDa (lane 2) to a thick band of over 250 kDa in the conjugates (lanes 3-4) resulting from the covalent addition of a random number of 115 kDa CPS chains to the protein. FIG. 1C: Western Blot using an anti-CPS mAb. Depolymerized CPS included as a control in all gels (lane 5). FIG. 1D: Control staining using an anti-TT mAb indicates preservation of the antigenicity of TT in the conjugates. It should be noted that differences in signal intensities between the 2:1 and 1:1 conjugate preparations (FIG. 1 A-D, lanes 3-4) are likely related to the total amounts of protein content (4.5 μg vs. 6.3 μg, respectively) within the 10 μg loaded sample per lane. FIG. 1E: Depolymerization of S. suis type 2 capsular polysaccharide (CPS) by ultrasonic irradiation. Samples of CPS were taken at different time points and were analyzed by size-exclusion chromatography coupled with multi-angle light scattering (SEC-MALS) in order to determine the molecular weight (M_{w}). After 60 min, the M_{w} plateaued, as illustrated by the dotted line.

FIG. 2: HPLC analysis demonstrating the elution of the conjugate (>250 kDa), elution of free CPS (100 kDa) and free TT (150 kDa).

FIG. 3A-3C: Kinetics of total antibody responses of mice immunized with 25 μg of the 2:1 conjugate vaccine adjuvanted with either CpG (FIG. 3A); STIMUNE® (FIG. 3B), or TITERMAX® Gold (FIG. 3C). Mice (n=10) were immunized on day 0 and boosted on day 21. ELISA plates were coated either with native capsular polysaccharide (CPS) or tetanus toxoid (TT) and incubated with blood samples diluted 1:100 or 1:20,000 to measure anti-CPS and anti-TT antibodies, respectively. Total (IgG+IgM) antibody levels are shown for individual mice, with horizontal bars representing mean±SEM of O.D. 450 nm values. Arrow at day 21 indicates boost. To simplify the graph, kinetics for the respective placebo groups are shown in FIG. 3D-3F. FIG. 3D-3F: Mice immunized with adjuvant only did not show any non-specific antibody response. Placebo groups were injected with PBS adjuvanted with either CpG (FIG. 3D), STIMUNE® (FIG. 3E), or TITERMAX® Gold (FIG. 3F). Mice (n=5) were injected on day 0 and boosted on day 21. ELISA plates were coated either with native capsular polysaccharide (CPS) or tetanus toxoid (TT) and incubated with blood samples diluted 1:100 or 1:20,000 to measure anti-CPS and anti-TT antibodies, respectively. Kinetics of total (IgG+IgM) antibody levels are shown for individual mice, with horizontal bars representing mean±SEM of O.D. 450 nm values. Arrow at day 21 indicates boost.

FIG. 4A-4H: Dose-response effect on total antibody levels of mice immunized with either free depolymerized capsular polysaccharide (CPS) at 1 μg, 2.5 μg, 5.0 μg, or 25 μg, respectively (FIG. 4A-4D); or with the 2:1 conjugate mix adjuvanted with TITERMAX® Gold at 1 μg, 2.5 μg, 5.0 μg, or 25 μg, respectively (FIG. 4E-4H). Mouse groups (n=8) were injected on day 0 and boosted on day 21. ELISA plates were coated with native CPS and incubated with blood samples diluted 1:100. Total (IgG+IgM) anti-CPS antibody levels are shown for individual mice, with horizontal bars representing mean±SEM of O.D._{450 nm} values. Arrow at day 21 indicates boost.

FIG. 5A-5F: Titers of different anti-CPS antibody isotypes in mice immunized with conjugate vaccines adjuvanted in TITERMAX® Gold. FIG. 5A. Murine Ig[G+M]; FIG. 5B. Murin IgG1; FIG. 5C. Murine IgG2c; FIG. 5D. Murine IgM; FIG. 5E. Murine IgG2b; FIG. 5F. Murine IgG23. Isotypes were detected using specific HRP-conjugated anti-mouse Ig[G+M], IgM, IgG1, IgG2b, IgG2c or IgG3 antibodies, respectively. Titers for individual mice are shown, with horizontal bars representing mean±SEM. # denotes titers significantly different than those of the placebo group (P<0.05), while differences between other groups are denoted as: **, P<0.01 and ***, P<0.001. Mouse groups were as follows: placebo (n=5), 2:1 conjugate formulation (n=18, animals from the 2 previous immunizations with 25 μg), 1:1 conjugate formulation (n=10), 2:1 conjugate HPLC-fraction (n=10), 2 CPS: 1 TT unconjugated control mixture (n=10). All mice were immunized with 25 μg of antigen in TITERMAX® Gold on day 0, boosted on day 21 and sera collected at day 42. A pool of hyperimmune mouse sera from 6 mice was also included for comparative purposes. For the titration, ELISA plates were coated with native CPS and incubated with two-fold serial dilutions of sera.

FIGS. 6A-6C: FIG. 6A: Opsonophagocytosis killing of S. suis type 2 strain S735 by day 42-sera from mice immunized with different CPS conjugate vaccines adjuvanted with TITERMAX® Gold. Mouse groups were as follows: placebo (n=5), 2:1 conjugate formulation (n=10), 2:1 conjugate HPLC-fraction (n=10), 2 CPS: 1 TT unconjugated control mixture (n=10). Results are expressed as % of bacterial killing for individual mice, with horizontal bars representing mean±SEM. # denotes values significantly different than those of the placebo group (P<0.01), while differences between other groups are denoted as: ***, P<0.001. FIG. 6B: Isotyping of antibodies induced in mice immunized with 2:1 conjugate vaccine in STIMUNE®. Mice (n=10) were immunized on day 0 and boosted on day 21 with 25 μg of the 2:1 conjugate formulation adjuvanted with STIMUNE®. Placebo mice (n=5) were similarly injected with PBS adjuvanted with STIMUNE®. Sera were collected on day 42. FIG. 6C: Opsonophagocytosis killing of S. suis type 2 strain S735 by day 42-sera from mice immunized with 25 μg of the 2:1 conjugate formulation adjuvanted with STIMUNE®. Results are expressed as % of bacterial killing for individual mice, with horizontal bars representing mean±SEM.

FIG. 7A-7B: Immunogenicity and protection studies in pigs. Animals were blocked by litter and then randomly assigned to one of four groups: group 1, n=14; group 2, n=10, group 3, n=15; and group 4, n=5. Groups 1-4 were commingled until group 4 (strict control) was removed at study day 35. Blood samples were collected on study days 0, 21 and 34 for determination of serum antibody levels. The piglets were injected intramuscularly twice at a 3-week interval (study day 0 and 21) with 2 ml of the respective vaccine or placebo adjuvanted with STIMUNE®: group 1 was vaccinated with adjuvanted S. suis type 2 bacterin, group 2 was injected with the adjuvanted 2:1 conjugate vaccine, group 3 was given 2 ml of adjuvanted PBS. FIG. 7A: Kinetics of serum antibody response of immunized pigs. ELISA plates were coated with native capsular polysaccharide, incubated for 1 h with two-fold serial dilutions of sera, and isotypes were detected using specific HRP-conjugated anti-pig Ig[G+M] or IgG1 antibodies. Antibody titers for individual pigs are shown, with horizontal bars representing mean±SEM. Arrow at day 21 indicates boost. **, P<0.01 and ***, P<0.001 as determined by one-way ANOVA. FIG. 7B: Protection study. On day 36, groups 1-3 were challenged intraperitoneally with 3×10⁹ CFU/dose of S. suis type 2 isolate ATCC 700794. Following challenge, pigs were monitored daily over a period of seven days for the presence of clinical signs. Note: on day 21, one animal from the bacterin group was euthanized due to complications following serum collection, which leaves (n=14) at day 34 and for the challenge. **, P<0.01 for both bacterin- and 2:1 conjugate-vaccinated groups compared to placebo (challenge control) group.

### DETAILED DESCRIPTION

The invention provides an immunogenic composition, comprising: a capsular polysaccharide-protein conjugate, together with a physiologically acceptable vehicle, wherein said conjugate comprises a capsular polysaccharide from Streptococcus suis conjugated to a carrier protein, wherein in said capsular polysaccharides are prepared from Streptococcus suis serotypes 1, 2, 7 or 9, or any other serotype, or combinations thereof.

In yet another embodiment the immunogenic composition comprises a carrier protein selected from the group comprising native or inactivated bacterial toxins, bacterial outer membrane proteins, ovalbumin, keyhole limpet hemocyanin (KLH), bovine serum albumin (BSA), tuberculin.

In yet another embodiment the carrier protein is an inactivated bacterial toxin selected of the group comprising tetanus toxoid, diphtheria toxoid, non-toxic cross-reactive material of diphtheria toxin (CRM197), pertussis toxoid, cholera toxoid, E. coli LT, E. coli ST, and exotoxin A from Pseudomona aeruginosa, any other typical protein carrier used in humans, or any immunogenic peptide/fragments derived from the above.

In yet another embodiment the carrier protein is a S. suis-derived immunogenic somatic and/or secreted protein selected from, but not restricted to, the group comprising suilysin, MRP, EF, enolase, subtilisin, and DNAse.

In one preferred embodiment, a capsular polysaccharide from Streptococcus suis, prepared from Streptococcus suis serotypes 1, 2, 7 or 9, or any other serotype, or combinations thereof, is conjugated to the carrier protein tetanus toxoid.

One embodiment of the invention is a multivalent immunogenic composition, comprising: polysaccharide-protein conjugates prepared from at least two different S. suis serotypes, together with a physiologically acceptable vehicle, wherein each conjugate comprises a capsular polysaccharide from Streptococcus suis conjugated to a carrier protein, wherein in said capsular polysaccharides are prepared from Streptococcus suis serotypes 1, 2, 7 and/or 9 or any other serotype.

In yet another embodiment, the multivalent immunogenic composition prepared from at least two different S. suis serotypes, is conjugated to a carrier protein wherein said carrier protein is selected from the group comprising tetanus toxoid, diphtheria toxoid, non-toxic cross-reactive material of diphtheria toxin (CRM197), pertussis toxoid, cholera toxoid, E. coli LT, E. coli ST, and exotoxin A from Pseudomona aeruginosa, any other typical protein carrier used in humans, or any immunogenic peptide/fragments derived from the above.

In yet another embodiment, the multivalent immunogenic composition prepared from at least two different S. suis serotypes, is conjugated to a carrier protein wherein said carrier protein is selected from the group comprising a S. suis-derived immunogenic somatic and/or secreted protein selected from, but not restricted to, the group comprising suilysin, MRP, EF, enolase, subtilisin, and DNAse.

In another embodiment, the multivalent immunogenic prepared from at least two different S. suis serotypes, is conjugated or a carrier protein, wherein each capsular polysaccharide is separately conjugated to tetanus toxoid carrier protein.

Another embodiment of the invention comprises a method of reducing clinical signs of S. suis associated infection, including, but not limited to, impaired behavior, lameness, frequency of brain lesions and central nervous system-associated clinical signs, bacteremia, recovery and/or colonization of bacterium from internal tissues, inflammation in thoracic and abdominal cavities, and mortality in swine comprising the administration of an immunogenic composition comprising: a capsular polysaccharide-protein conjugate, together with a physiologically acceptable vehicle, wherein said conjugate comprises a capsular polysaccharide from Streptococcus suis conjugated to a carrier protein, wherein in said capsular polysaccharides are prepared from Streptococcus suis serotypes 1, 2, 7 or 9, or any other serotype or combinations thereof, to an animal in need thereof.

In a preferred embodiment a method of reducing clinical signs of S. suis associated infection, including, but not limited to, impaired behavior, lameness, frequency of brain lesions and central nervous system-associated clinical signs, bacteremia, recovery and/or colonization of bacterium from internal tissues, inflammation in thoracic and abdominal cavities, and mortality in swine comprises the administration of the immunogenic composition comprising a capsular polysaccharide from Streptococcus suis, prepared from Streptococcus suis serotypes 1, 2, 7 or 9, or any other serotype or combinations thereof, conjugated to the carrier protein tetanus toxoid, to an animal in need thereof.

In yet another embodiment a method of reducing clinical signs of S. suis associated infection, including, but not limited to, impaired behavior, lameness, frequency of brain lesions and central nervous system-associated clinical signs, bacteremia, recovery and/or colonization of bacterium from internal tissues, inflammation in thoracic and abdominal cavities, and mortality in swine comprises the administration of an immunogenic composition comprising a multivalent immunogenic composition, comprising: polysaccharide-protein conjugates prepared from at least two different S. suis serotypes, together with a physiologically acceptable vehicle, wherein each conjugate comprises a capsular polysaccharide from Streptococcus suis conjugated to a carrier protein, wherein in said capsular polysaccharides are prepared from Streptococcus suis serotypes 1, 2, 7 or 9, or any other serotype to an animal in need thereof.

An embodiment of the invention also comprises a method for making an immunogenic conjugate comprising: a Streptococcus suis serotype 1, 2, 7 and/or 9 or any other serotype capsular polysaccharide, or combinations thereof, covalently linked to a carrier protein, the method comprising: (a) depolymerizing capsular polysaccharides of S. suis serotype 1, 2, 7, and/or 9 or any other serotype by sonication or phage degradation, mild acid hydrolysis or ozonation; (b) reacting depolymerized capsular polysaccharides (CPS) of step (a) with sodium periodate to yield <10% oxidation levels (or any other oxidation level without loss of immunogenicity) of sialic acid residues or any other target sugar residue by chemical or enzymatic oxidation, such as galactose oxidase and related enzymes capable of specifically modifying particular sugars being part of the CPS; (c) covalently coupling the periodate treated capsular polysaccharides (CPS) of step (b) to a carrier protein by reductive amination or any other method of conjugation known in the art of CPS-protein conjugate vaccines, resulting in polysaccharide:carrier protein conjugates; and (d) reacting the polysaccharide:carrier protein conjugates to reduce free aldehyde groups; wherein the resulting CPS:carrier protein ratio is 2:1 or 1:1 or any other ratios that allow preserving the immunogenicity of either or both the CPS or the protein carrier.

In one embodiment, the method for making an immunogenic conjugate comprises Streptococcus suis serotype 1, 2, 7 and/or 9 or any other serotype capsular polysaccharides, or combinations thereof, covalently linked to a carrier protein, wherein said carrier protein is selected from the group comprising inactivated bacterial toxins, bacterial outer membrane proteins, ovalbumin, keyhole limpet hemocyanin (KLH), bovine serum albumin (BSA), or tuberculin.

In yet another embodiment, the method for making an immunogenic conjugate comprises Streptococcus suis serotype 1, 2, 7 and/or 9 or any other serotype capsular polysaccharides, or combinations thereof, covalently linked to a carrier protein, wherein said carrier protein is an inactivated bacterial toxin selected of the group comprising tetanus toxoid, diphtheria toxoid, non-toxic cross-reactive material of diphtheria toxin (CRM197), pertussis toxoid, cholera toxoid, E. coli LT, E. coli ST, and exotoxin A from Pseudomona aeruginosa, any other typical protein carrier used in humans, or any immunogenic peptide/fragments derived from the above

In yet another embodiment, the method for making an immunogenic conjugate comprises Streptococcus suis serotype 1, 2, 7 and/or 9 capsular polysaccharides, or any other serotype, or combinations thereof, covalently linked to a carrier protein, wherein said carrier protein is a S. suis-derived immunogenic somatic and/or secreted protein selected from, but not restricted to, the group comprising suilysin, MRP, EF, enolase, subtilisin, and DNAse

In a preferred embodiment, the method for making an immunogenic conjugate comprises Streptococcus suis serotype 1, 2, 7 and/or 9 or any other serotype capsular polysaccharides, or combinations thereof, covalently linked to a carrier protein, wherein said carrier protein is tetanus toxoid.

The practice of the present invention will employ, unless otherwise indicated, conventional techniques of molecular biology, microbiology, recombinant DNA technology, protein and polysaccharide chemistry and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); Protein purification methods—a practical approach (E. L. V. Harris and S. Angal, eds., IRL Press at Oxford University Press); and Handbook of Experimental Immunology, Vols. I-IV (D. M. Weir and C. C. Blackwell eds., 1986, Blackwell Scientific Publications); R. Roy in: Carbohydrate-based vaccines, ACS Symposium Series, 989, 2008.

Before describing the present invention in detail, it is to be understood that this invention is not limited to particular DNA, polypeptide sequences or process parameters as such may, of course, vary. It is also to be understood that the terminology used herein is for the purpose of describing particular embodiments of the invention only, and is not intended to be limiting. It must be noted that, as used in this specification and the appended claims, the singular forms “a”, “an” and “the” include plural referents unless the content clearly dictates otherwise. Thus, for example, reference to “an antigen” includes a mixture of two or more antigens, reference to “an excipient” includes mixtures of two or more excipients, and the like.

### A. Definitions

Unless defined otherwise, all technical and scientific terms used herein have the same meaning as is commonly understood by one of skill in the art to which this invention belongs at the time of filing. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular. Herein, the use of “or” means “and/or” unless stated otherwise. Furthermore, the use of the term “including”, as well as other forms such as “includes” and “included” is not limiting. All patents and publications referred to herein are incorporated by reference herein.

“Protection against disease”, “protective immunity”, “functional immunity” and similar phrases, means a response against a disease or condition generated by administration of one or more therapeutic compositions of the invention, or a combination thereof, that results in fewer deleterious effects than would be expected in a non-immunized subject that has been exposed to disease or infection. That is, the severity of the deleterious effects of the infection is lessened in a vaccinated subject. Infection may be reduced, slowed, or possibly fully prevented, in a vaccinated subject. Herein, where complete prevention of infection is meant, it is specifically stated. If complete prevention is not stated then the term includes partial prevention.

Herein, “reduction of the incidence and/or severity of clinical signs” or “reduction of clinical symptoms” means, but is not limited to, reducing the number of infected subjects in a group, reducing or eliminating the number of subjects exhibiting clinical signs of infection, or reducing the severity of any clinical signs that are present in one or more subjects, in comparison to wild-type infection. For example, it should refer to any reduction of pathogen load, pathogen shedding, reduction in pathogen transmission, or reduction of any clinical sign symptomatic of Streptococcus suis infection. Preferably these clinical signs are reduced in one or more subjects receiving the therapeutic composition of the present invention by at least 10% in comparison to subjects not receiving the composition and that become infected. More preferably clinical signs are reduced in subjects receiving a composition of the present invention by at least 20%, preferably by at least 30%, more preferably by at least 40%, and even more preferably 50%, and even more preferably 70%.

The term “increased protection” herein means, but is not limited to, a statistically significant reduction of one or more clinical symptoms which are associated with infection by an infectious agent, preferably S. suis, respectively, in a vaccinated group of subjects vs. a non-vaccinated control group of subjects. The term “statistically significant reduction of clinical symptoms” means, but is not limited to, the frequency in the incidence of at least one clinical symptom in the vaccinated group of subjects is at least 10%, preferably 20%, more preferably 30%, even more preferably 50%, and even more preferably 70% lower than in the non-vaccinated control group after the challenge with the infectious agent.

“Long-lasting protection” shall refer to “improved efficacy” that persists for at least 3 weeks, but more preferably at least 3 months, still more preferably at least 6 months. In the case of livestock, it is most preferred that the long lasting protection shall persist until the average age at which animals are marketed for meat.

An “immunogenic or immunological composition” refers to a composition of matter that comprises at least one bacterial capsular polysaccharide-protein conjugate that elicits an immunological response in the host of a cellular or antibody-mediated immune response to the composition. In a preferred embodiment of the present invention, an immunogenic composition induces an immune response and, more preferably, confers protective immunity against one or more of the clinical signs of a S. suis infection.

An “immunogenic” bacterial capsular polysaccharide-protein conjugate, or “antigen” as used herein refer to a polysaccharide coupled to a protein carrier that elicits an immunological response as described herein. An “immunogenic” bacterial capsular polysaccharide-protein conjugate includes polysaccharides derived from S. suis serotypes 1, 2, 7, and 9 or any other serotype wherein the (poly)saccharide is obtained by synthetic means known for those skilled in the art or is depolymerized prior to conjugation to the protein carrier, to a molecular weight ranging from 100-400 kDa. For example, in one aspect, the molecular weight ranges from 100-to 350 kDa, from 100 to 300 kDa, from 100 to 250 kDa, from 100 to 200 kDa, from 100 to 150 kDa, from 200 to 400 kDa, from 200 to 350 KDa, from 200 to 300 kDa, from 200 to 250 kDa, from 300 to 400 kDa, or from 300 to 350 kDa, or from 5 to 400 kDa or as synthetic oligosaccharides fragments thereof. In one embodiment the carrier protein covalently coupled to the polysaccharide is a toxoid from tetanus, diphtheria, pertussis, Pseudomonas, E. coli, Staphylococcus, Streptococcus, Clostridium perfringens, or Salmonella, or any immunogenic peptide/fragments derived from the above. The size of the CPS or its synthetic fragments together with the protein ratios being optimized for the best immunogenic composition, usually composed of a CPS of 5 kDa or higher and ratios of 4-5 CPS (fragments); 1 protein (or peptide fragments).

The term “conjugate” as used herein refers to a polysaccharide covalently conjugated to a carrier protein. Conjugates of the disclosure and immunogenic composition comprising them may contain some amount of free (non-covalently linked) polysaccharide and free carrier protein.

As used herein, “to conjugate,” “conjugated” and conjugating” refer to a process whereby a polysaccharide or bacterial capsular polysaccharide, is covalently attached to a carrier molecule or carrier protein. The conjugation can be performed according to the methods described below or by other processes known in the art. Conjugation enhances the immunogenicity of the capsular polysaccharide.

The term “saccharide” as used herein is used interchangeably with “polysaccharide”, or “oligosaccharide” to refer to bacterial capsular polysaccharides, in one preferred embodiment isolated from S. suis.

An “immune response” or “immunological response” means, but is not limited to, the development of a cellular and/or antibody-mediated immune response to the composition or vaccine of interest. Usually, an immune or immunological response includes, but is not limited to, one or more of the following effects: the production of antibodies, the activation of B cells, helper T cells, and/or cytotoxic T cells, directed specifically to an antigen or antigens included in the composition or vaccine of interest. Preferably, the host will display either a therapeutic or a protective immunological (memory) response such that resistance to new infection will be enhanced and/or the clinical severity of the disease reduced. Such protection will be demonstrated by either a reduction in number of symptoms, severity of symptoms, or the lack of one or more of the symptoms associated with the infection of the pathogen, a delay in the of onset of clinical signs of S. suis associated infection, including, but not limited to, impaired behavior, lameness, frequency of brain lesions and central nervous system-associated clinical signs, bacteremia, recovery and/or colonization of bacterium from internal tissues, inflammation in thoracic and abdominal cavities, and mortality

As used herein, “a pharmaceutical- or veterinary-acceptable carrier” includes any and all solvents, dispersion media, coatings, adjuvants, stabilizing agents, diluents, preservatives, antibacterial and antifungal agents, isotonic agents, adsorption delaying agents, and the like. In some preferred embodiments, and especially those that include lyophilized immunogenic compositions, stabilizing agents for use in the present invention include stabilizers for lyophilization or freeze-drying.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

“Isolated” means altered “by the hand of man” from its natural state, i.e., if it occurs in nature, it has been changed or removed from its original environment, or both. For example, a bacterial capsular polysaccharide naturally present in a living organism is not “isolated,” but the same capsular polysaccharide separated from the coexisting materials of its natural state is “isolated”, as the term is employed herein.

The terms “vaccination” or “vaccinating” or variants thereof, as used herein means, but is not limited to, a process which includes the administration of an immunogenic composition of the invention that, when administered to an animal, elicits, or is able to elicit—directly or indirectly—, an immune response in the animal against S. suis.

“Mortality”, in the context of the present invention, refers to death caused by S. suis infection, and includes the situation where the infection is so severe that an animal is euthanized to prevent suffering and provide a humane ending to its life.

Herein, “effective dose” means, but is not limited to, an amount of antigen that elicits, or is able to elicit, an immune response that yields a reduction of clinical symptoms in an animal to which the antigen is administered.

As used herein, the term “effective amount” means, in the context of a composition, an amount of an immunogenic composition capable of inducing an immune response that reduces the incidence of or lessens the severity of infection or incident of disease in an animal. Alternatively, in the context of a therapy, the term “effective amount” refers to the amount of a therapy which is sufficient to reduce or ameliorate the severity or duration of a disease or disorder, or one or more symptoms thereof, prevent the advancement of a disease or disorder, cause the regression of a disease or disorder, prevent the recurrence, development, onset, or progression of one or more symptoms associated with a disease or disorder, or enhance or improve the prophylaxis or treatment of another therapy or therapeutic agent.

B. Carrier Molecules

The carrier molecules to which the S. suis capsular polysaccharides of the invention can be conjugated or covalently linked are preferably those described above. Preferred carriers include, but are not limited to inactivated bacterial toxins, such as a toxoid from tetanus, diphtheria, pertussis, Pseudomonas, E. coli, Staphylococcus, Streptococcus, Clostridium perfringens, or Salmonella; or bacterial outer membrane proteins, ovalbumin, keyhole limpet hemocyanin (KLH), bovine serum albumin (BSA), or tuberculin; or S. suis-derived immunogenic somatic and/or secreted protein selected from, but not restricted to, the group comprising suilysin, MRP, EF, enolase, subtilisin, DNAse; or any immunogenic peptide/fragments derived from the above and not limited to (such as PADRE®). Preferably, the carrier protein itself is an immunogen.

The S. suis capsular polysaccharides of the invention can be prepared by standard techniques known to those skilled in the art. For example capsular polysaccharides can be prepared from a variety of S. suis serotypes, including, but not limited to serotypes 1, 2, 7, and 9. The individual polysaccharides are purified through centrifugation, precipitation, ultra-filtration, and gel filtration/size exclusion chromatography; and then depolymerized by sonication or phage degradation, mild acid hydrolysis or ozonation; or, alternatively, individual oligosaccharides can be obtained by synthetic means known for those skilled in the art. The purified/depolymerized/synthetized poly(oligo)saccharides are chemically activated to make them reactive with the carrier protein. Once activated each capsular polysaccharide is conjugated to a carrier protein to form a “S. suis capsular polysaccharide-protein conjugate”.

S. suis capsular polysaccharides may be covalently coupled to the carrier by any convenient method known to the art (R. Roy, Carbohydrate-based vaccines, ACS Symp. Ser, 989, 2008). For example, the present disclosure provides methods comprise (1) isolating the capsular polysaccharide; (2) depolymerizing the polysaccharide; (3) activating the polysaccharide; (4) reacting the activated polysaccharide with a carrier protein wherein the end product is stable polysaccharide-protein conjugate. In the one embodiment the capsular polysaccharide is depolymerized by sonication or, alternatively, by phage degradation, mild acid hydrolysis or ozonation, wherein the after depolymerization molecular weight was determined by size-exclusion chromatography. Depolymerized polysaccharide is then activated in the presence of an oxidizing agent, in a non-limiting example, the oxidizing agent is sodium or usual alkali periodates, or any other chemical or enzymatic oxidation of any target sugar residue. The degree of oxidation of the sialic acid or other sugar residues is assessed by gas chromatography/HPLC-MS. Treated polysaccharides are coupled by reductive amination in the presence of, but not limited to, sodium cyanoborohydride in controlled buffers for a 2:1 or 1:1 conjugate ratio, or any ratios being optimized for the best immunogenic composition, usually composed of a CPS of 5 kDa or higher and ratios of 4-5 CPS (fragments); 1 protein (or peptide fragments).

The size of the immunogenic composition, as defined by average molecular weight, is variable and dependent upon the chosen bacterial capsular polysaccharide derived from S. suis serotypes 1, 2, 7, or 9, or any other serotype, the protein carrier, and the method of depolymerization and the method of coupling of the bacterial capsular polysaccharides to the carrier. Therefore, it can be as small as 1,000 Daltons (10³) or greater than 10⁶ Daltons.

Toxicity and therapeutic efficacy of such molecules can be determined by standard pharmaceutical procedures in cell cultures or experimental animals, e.g., for determining the LD₅₀ (the dose lethal to 50% of the population).

The vaccines of the invention may be multivalent or monovalent. Multivalent vaccines are made from immuno-conjugation of multiple bacterial capsular polysaccharides derived from S. suis serotypes 1, 2, 7, or 9, or any other serotype with a carrier molecule.

In yet another aspect, the bacterial capsular polysaccharide-protein conjugate compositions comprise an effective immunizing amount of the immunogenic conjugate, in combination with an additional immunostimulant; and a physiologically acceptable vehicle. As used in the present context, “immunostimulant” is intended to encompass any compound or composition which has the ability to enhance the activity of the immune system, whether it is a specific potentiating effect in combination with a specific antigen, or simply an independent effect upon the activity of one or more elements of the immune response. Immunostimulant compounds include but are not limited to mineral gels, e.g., aluminum hydroxide; surface active substances such as lysolecithin, pluronic polyols; polyanions; peptides; oil emulsions; and MDP. Methods of utilizing these materials are known in the art, and it is well within the ability of the skilled artisan to determine an optimum amount of stimulant for a given vaccine. More than one immunostimulant may be used in a given formulation. The immunogen (CPS) may also be non-covalently incorporated in micellar or liposomal compositions for use in a vaccine formulation.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration preferably for administration to a mammal, especially a pig. Associated with such container(s) can be a notice in the form prescribed by a governmental agency regulating the manufacture, use or sale of pharmaceuticals or biological products, which notice reflects approval by the agency of manufacture, use or sale for human administration.

### C. Adjuvants

In order to further increase the immunogenicity of the immunogenic compositions provided herewith, and which contain one or more S. suis capsular polysaccharide-protein conjugates may also comprise one or more adjuvants.

In some embodiments, the immunogenic composition of the present invention contains an adjuvant. “Adjuvants” as used herein, can include, for example aluminum hydroxide and aluminum phosphate, saponins [e.g., Quil A, QS-21 (Cambridge Biotech Inc., Cambridge Mass.)], GPI-0100 (Galenica Pharmaceuticals, Inc., Birmingham, Ala.), water-in-oil emulsions, oil-in-water emulsions, water-in-oil-in-water emulsions [e.g., water-in-oil formulations, including TITERMAX® Gold (Sigma-Aldrich, St. Louis, Mo.), and STIMUNE® (Specol, LifeTechnologies)]. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.), JohnWiley and Sons, NY, pp 51-94 (1995) and Todd et al., Vaccine 15:564-570 (1997). Exemplary adjuvants are the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Carbopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide, CpG ODN a synthetic version of bacterial oligonucleotide [e.g., ODN 1826 VACCIGRADE™ (InvivoGen, San Diego, Calif.)], or naturally occurring or recombinant cytokines or analogs thereof or stimulants of endogenous cytokine release, among many others.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 0.01 to 65%, preferably at a concentration of about 2% to 30%, more preferably at a concentration of about 5% to 25%, still more preferably at a concentration of about 7% to 22%, and most preferably at a concentration of 10% to 20% by volume of the final product. The vaccine compositions of the invention are prepared by physically mixing the adjuvant with the S. suis capsular polysaccharide-protein conjugates under appropriate sterile conditions in accordance with known techniques to produce the adjuvanted composition.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 20% to 65%, preferably at a concentration of about 20% to 30%, more preferably at a concentration of about 5% to 25%, still more preferably at a concentration of about 7% to 22%, and most preferably at a concentration of 10% to 20% by volume of the final product.

### D. Physiologically-Acceptable Vehicles

The vaccine compositions of this invention may be formulated using techniques similar to those used for other pharmaceutical polypeptide compositions. Thus, the adjuvant and S. suis capsular polysaccharide-protein conjugates, may be stored in lyophilized form and reconstituted in a physiologically acceptable vehicle to form a suspension prior to administration. Alternatively, the adjuvant and conjugate may be stored in the vehicle. Preferred vehicles are sterile solutions, in particular, sterile buffer solutions, such as phosphate buffered saline. Any method of combining the adjuvant and the conjugate in the vehicle such that improved immunological effectiveness of the immunogenic composition is appropriate.

The volume of a single dose of the vaccine of this invention may vary but will be generally within the ranges commonly employed in conventional vaccines. The volume of a single dose is preferably between about 0.1 ml and about 3 ml, preferably between about 1.0 ml and about 3.0 ml, and more preferably between about 1.0 ml and about 2.0 ml at the concentrations of conjugate and adjuvant noted above.

The vaccine compositions of the invention may be administered by any convenient means.

### E. Formulation

Immunogenic conjugates comprising a S. suis capsular polysaccharides coupled to a carrier molecule can be used as vaccines for immunization against one or more serotypes of S. suis, including but not limited to, serotypes 1, 2, 7, and 9. The vaccines, comprising the immunogenic conjugate in a physiologically acceptable vehicle, are useful in a method of immunizing animals, preferably swine, for prevention of infections by S. suis.

Antibodies generated against immunogenic conjugates of the present invention by immunization with an immunogenic conjugate can be used in passive immunotherapy for preventing infections of S. suis.

The subject to which the composition is administered is preferably a swine. In another embodiment the subject is a human.

The formulations of the invention comprise an effective immunizing amount of one or more immunogenic compositions or antibodies thereto and a physiologically acceptable vehicle. Vaccines comprise an effective immunizing amount of one or more immunogenic compositions and a physiologically acceptable vehicle. The formulation should suit the mode of administration.

The immunogenic composition, if desired, can also contain minor amounts of wetting or emulsifying agents, or pH buffering agents. The immunogenic composition can be a liquid solution, suspension, emulsion, capsule, sustained release formulation.

### F. Effective Dose

The compounds described herein can be administered to a subject at therapeutically effective doses to prevent S. suis associated diseases. The dosage will depend upon the host receiving the vaccine as well as factors such as the age of the host.

The precise amount of immunogenic conjugate or antibody of the invention to be employed in a formulation will depend on the route of administration and the nature of the subject (e.g., species, age, size,), and will be demonstrated in efficacy studies as required by the governing regulatory agencies.

Toxicity and therapeutic efficacy of compounds can be determined in experimental animals. While compounds that exhibit toxic side effects can be used, care should be taken to design a delivery system that targets such compounds to the site of affected tissue in order to minimize potential damage to uninfected cells and, thereby, reduce side effects.

The data obtained from and animal studies can be used in formulating a range of dosage for use swine. The dosage can vary within this range depending upon the dosage form employed and the route of administration utilized.

Immunogenicity of a composition can be determined by monitoring the immune response of test subjects following immunization with the composition by use of any immunoassay known in the art. Generation of a humoral (antibody) response and/or cell-mediated immunity may be taken as an indication of an immune response. Test subjects may include animals such as pigs, mice, hamsters, dogs, cats, rabbits, cows, horses, sheep, poultry (e.g. chickens, ducks, geese, and turkeys), and humans.

The immune response of the test subjects can be analyzed by various approaches such as: the reactivity of the resultant immune serum to the immunogenic conjugate, as assayed by known techniques, e.g., enzyme linked immunosorbent assay (ELISA), immunoblots, immunoprecipitations, etc.; or, by protection of immunized hosts from infection by the pathogen and/or attenuation of symptoms due to infection by the pathogen in immunized hosts as determined by any method known in the art, for assaying the levels of an infectious disease agent, e.g., the bacterial levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use in swine.

### G. Administration to a Subject

Preferred routes of administration include but are not limited to intranasal, oral, intradermal, and intramuscular. Administration in drinking water, most preferably in a single dose, is desirable. The skilled artisan will recognize that compositions of the invention may also be administered in one, two or more doses, as well as, by other routes of administration. For example, such other routes include subcutaneously, intracutaneously, intravenously, intravascularly, and intracardially. Depending on the desired duration and effectiveness of the prophylaxis, the compositions according to the invention may be administered once or several times, also intermittently, for instance on a daily basis for several days, weeks or months, bi-annually, or yearly intervals and in different dosages.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

Bacterial Strains and Growth Conditions:

S. suis serotype 2 reference strain S735 (ATCC 43765) was used as the source of type 2 CPS (Van Calsteren et al. Biochem Cell Biol. 2010; 88:513-25), as the target strain for in vitro opsonophagocytic assays (OPA), and to prepare the heat-killed bacteria used to hyperimmunize mice. Isolated colonies on sheep blood agar plates were inoculated in 5 ml of Todd-Hewitt Broth (THB; Oxoid, Nepean, ON, Canada) and incubated for 8 h in a water bath at 37° C. with 120 rpm agitation. Working cultures were prepared by transferring 10 μl of 8 h-cultures diluted 1:1,000 with PBS into 30 ml of THB which was incubated for 16 h. Bacteria were washed once and resuspended in PBS to obtain 5×10⁸ CFU/ml. Heat-killed bacterial cultures were obtained as previously described (Segura, et al. Infect Immun. 1999; 67:4646-54). Briefly, overnight cultures were washed once with PBS, and then resuspended in 30 ml of fresh THB. A sample was taken to perform bacterial counts on THB Agar (THA). Bacteria were immediately killed by incubating at 60° C. for 45 min, then cooled on ice. Bacterial killing was confirmed by absence of growth on blood agar for 48 h. Strains used for the swine challenge model are described below.

Isolation and Purification of Type 2 S. suis CPS:

Bacterial cultures were grown as described by Calzas et al. (Infect Immun. 2013; 81:3106-18). CPS extraction and purification, followed by quality controls comprising protein determination by the modified Lowry protein assay kit (Pierce, Rockford, Ill., USA), nucleic acid quantification using an ND-1000 spectrometer (Nanodrop, Wilmington, Del., USA) and 1D/2D ¹H nuclear magnetic resonance (NMR) analysis to ensure purity and identity were performed as described by Van Calsteren et al. (Biochem Cell Biol. 2010; 88:513-25).

Control Mouse Antiserum:

Hyperimmune mice (n=6) were obtained by repeated immunization of 5 week-old female C57BL/6 mice with 7.5×10⁸ CFU/ml heat-killed S. suis serotype 2 strain S735 in THB by intraperitoneal injection on days 0, 7, 21, and 28. On day 42, serum was collected, pooled, aliquoted, and stored at −80° C.

Measurement of Antibodies Against Type 2 S. suis CPS and TT:

To measure specific antibodies, 200 ng of either native S. suis serotype 2 CPS or TT in 0.1 M NaCO₃, pH 9.6, were added to wells of an ELISA plate (Nunc-Immuno Polysorp, Canadawide Scientific, Toronto, ON, Canada). After overnight coating at 4° C., plates were washed with PBS containing 0.05% (v/v) Tween 20 (PBST) and blocked by the addition of PBS containing 1% (w/v) of BSA (HyClone, Logan, Utah, USA) for 1 h. After washing, mouse blood or mouse/porcine serum samples diluted in PBST were added to the wells for 1 h. After washing, the plates were incubated for 1 h with a HRP-conjugated isotype specific antibody diluted in PBST as described below. The enzyme reaction was developed by addition of 3,3′,5,5′-tetramethylbenzidine (TMB; InvitroGen, Burlington, ON, Canada), stopped by addition of 0.5 M H₂SO₄, and the absorbance was read at 450 nm with an ELISA plate reader.

To follow the kinetics of total (IgG+IgM) antibody responses to CPS and TT, mouse blood collected from the tail vein was diluted 1:100 or 1:20,000, respectively. Dilution optimization had previously been conducted (data not shown). HRP-conjugated goat anti-mouse IgG+IgM (H+L) at a dilution of 1:2,500 (Jackson Immunoresearch) was used as detection antibody.

To perform the titration of mouse Ig isotypes, day 42-serum was serially diluted (two-fold) in PBST, and antibodies were detected using either HRP-conjugated goat anti-mouse IgG+IgM as aforementioned, goat anti-IgM diluted 1:1000, goat anti-IgG1, goat anti-IgG2b, goat anti-IgG2c or goat anti-IgG3 diluted 1:400 (Southern Biotech). For porcine serum, two-fold serial dilutions were performed in PBST and antibodies were detected using HRP-conjugated goat anti-swine total Ig [IgG+IgM] diluted 1:4,000 (Jackson Immunoresearch). To detect porcine IgG subclasses, unconjugated mouse anti-swine IgG1 or mouse anti-swine IgG2 (AbD serotec, Raleigh, N.C., USA) diluted 1:250 was added followed by incubation with HRP-conjugated goat anti-mouse secondary antibody. For both, mouse and pig serum titration, the reciprocal of the last serum dilution that resulted in an optical density (OD_{450 nm}) equal or lower of 0.2 (as a pre-established cutoff for comparison purposes) was considered the titer of that serum. For representation purposes, negative titers cutoff) were given an arbitrary titer value of 10.

To control inter-plate variations, an internal reference positive control was added to each plate. For titration of mouse antibodies, this control was a pool of sera from hyper-immunized mice (produced as described above). For titration of pig antibodies, this control was a serum of a pig hyper-immunized with 10⁸ CFU of a killed suspension of S. suis serotype 2. Reaction in TMB was stopped when an OD_{450 nm} of 1 was obtained for the positive internal control. Optimal dilutions of the coating antigen (CPS or TT), the positive internal control sera and the HRP-conjugated anti-mouse or anti-pig antibodies were determined during preliminary standardizations.

Opsonophagocytosis Assay:

Blood was collected by intracardiac puncture from naïve C57BL/6 mice, treated with sodium heparin, then diluted to obtain 6.25×10⁶ leukocytes/ml in RPMI 1640 supplemented with 5% heat-inactivated fetal bovine serum, 10 mM HEPES, 2 mM L-glutamine and 50 μM 2-mercaptoethanol. All reagents were from Gibco (InvitroGen, Burlington, ON, Canada). All blood preparations were kept at room temperature. Using washed bacterial cultures grown as described above, final bacterial suspensions were prepared in complete cell culture medium to obtain a concentration of 1.25×10⁶ CFU/ml. The number of CFU/ml in the final suspension was determined by plating samples onto THA using an Autoplate 4000 Automated Spiral Plater (Spiral Biotech, Norwood, Mass., USA). All bacterial preparations were kept on ice. Diluted whole blood at 5×10⁵ leukocytes was mixed with 5×10⁴ CFU of S. suis (multiplicity of infection [MOI] of 0.1) and 40% (v/v) of serum from naïve or vaccinated mice in a microtube to a final volume of 0.2 ml. The tube tops were pierced using a sterile 25G needle, then the microtubes were incubated for 2 h at 37° C. with 5% CO₂, with gentle manual agitation every 20 min. After incubation, viable bacterial counts were performed on THA using an Autoplate 4000 Automated Spiral Plater. Tubes with addition of naive rabbit serum or rabbit anti-S. suis type 2 strain S735 serum (Higgins and Gottschalk. J Vet Diagn Invest. 1990; 2:249-52) were used as negative and positive controls, respectively. The % of bacterial killing was determined using the following formula: % Bacteria killed=[1−(bacteria recovered from sample tubes/bacteria recovered from negative control tubes with naïve mouse sera)]×100. Final OPA conditions were selected based in several pre-trials using different incubation times and MOIs (Goyette-Desjardins et al. Methods Mol Biol. 2015; 1331:81-92).

Statistical Analyses:

All data are expressed as means±standard errors of the means (SEM). Data were analyzed for significance using analysis of variance (ANOVA) from SigmaPlot version 11.0, except for the survival curves analysis, which was performed using the log-rank test from GraphPad version 5.01. Significance is denoted in the figures as follows: *, P<0.05; **, P<0.01 and ***, P<0.001.

### Example 1

### Preparation of the Conjugate Vaccines:

### 1. Depolymerization of Type 2 CPS:

In 2010, Van Calsteren et al. reported the exact structure of the repeating unit for the serotype 2 CPS (Byrd and Kadis. Infect Immun. 1992; 60:3042-51). The CPS repeating unit is composed of a unique arrangement of 1 rhamnose:1 glucose:3 galactoses:1 N-acetylglucosamine:1 sialic acid (also named as N-acetylneuraminic acid [Neu5Ac]). The sialic acid is found to be terminal on a branch with an α2,6-linkage to a galactose. The precise knowledge of S. suis serotype 2 CPS structure provides the chemical bases for the construction of a glycoconjugate.

Using highly purified CPS from S. suis type 2 containing less than 1% w/w of proteins or nucleic acids as previously described by Calzas et al. (Calzas et al. Infect Immun. 2013; 81:3106-18), it was investigated whether conjugation to a carrier protein, such as TT, would circumvent the T cell-independent (TI) antigenicity of the CPS and instead induce a T cell-dependent (TD) protective humoral response in animals.

To produce the glycoconjugate, it was first decided to depolymerize the CPS to a smaller size in order to improve the efficacy of the conjugation and the residual exposure of the T cell peptide epitopes of the protein carrier. Due to its high M_{w}, found to be between 410,000-480,000 Da (Van Calsteren et al. Biochem Cell Biol. 2010; 88:513-25; and Calzas et al. Infect Immun 2013; 81:3106-18), the native polysaccharide of S. suis type 2 was first depolymerized into smaller fragments.

To perform this depolymerization, ultrasonic irradiation as described by Szu et al. (Szu et al. Carbohydr Res. 1986; 152:7-20) was conducted. Ultrasonic irradiation (sonication) was chosen over fragmentation by chemical (Duan and Kasper. Glycobiology 2011; 21:401-9; Higashi et al. Carbohydr Polym 2011; 86:1365-70; and Anderson P. Infect Immun 1983; 39:233-8) or enzymatic (Svenson et al. FEMS Microbiol Lett 1977; 1:145-7; Svenson et al. J Immunol Methods 1979; 25:323-35; Wessels et al. Proc Natl Acad Sci 1987; 84:9170-4; and Paoletti et al. J Biol Chem 1990; 265:18278-83) methods to avoid chemical alterations (Pawlowski and Svenson. FEMS Microbiol Lett 1999; 174:255-63). In addition, it is easy to use, reliable for labile epitopes and does not requires elimination of excess reagents. Another great advantage of ultrasonic irradiation is the reduction in sample polydispersity, resulting in a very narrow and homogenous distribution of M_{w} (Table 1) (Szu et al. 1986), facilitating biochemical characterization, particularly within the glycoconjugate.

Twenty milliliters of a 2 mg/ml solution of CPS in 50 mM NH₄HCO₃ were transferred to a 50 ml conical polypropylene tube in an ice-bath. A titanium ⅛ inch microtip probe mounted on a Virsonic 600 sonicator (Virtis, Gardiner, N.Y., USA) was immersed in the CPS solution, and sonication was performed at 20 kHz and 24 W for 60 min (see below). After sonication, a sample of CPS was taken to determine the molecular weight (M_{w}) by size-exclusion chromatography coupled with multi-angle light scattering (SEC-MALS) as previously described (Van Calsteren et al. Biochem Cell Biol 2013; 91:49-58), with some modifications. Briefly, the chromatographic separation was performed with two 8 mm×300 mm Shodex OHpak gel filtration columns connected in series (SB-806 and SB-804), preceded by a SB-807G guard column (Showa Denko, Tokyo, Japan). Elution was done at 0.5 ml/min using 0.1 M NaNO₃ as the mobile phase. Molar masses were determined using a Dawn EOS MALS detector (Wyatt, Santa Barbara, Calif., USA) and calculations were performed with the ASTRA software version 6.1.1.17 (Wyatt) using 11 detectors from angles 34.8° to 132.2° (detectors 5-15) for the depolymerized samples. The remaining solution of depolymerized CPS was dialyzed against water (Spectra/Por, MWCO 3,500; Spectrum Laboratories, Rancho Dominguez, Calif., USA) and lyophilized.

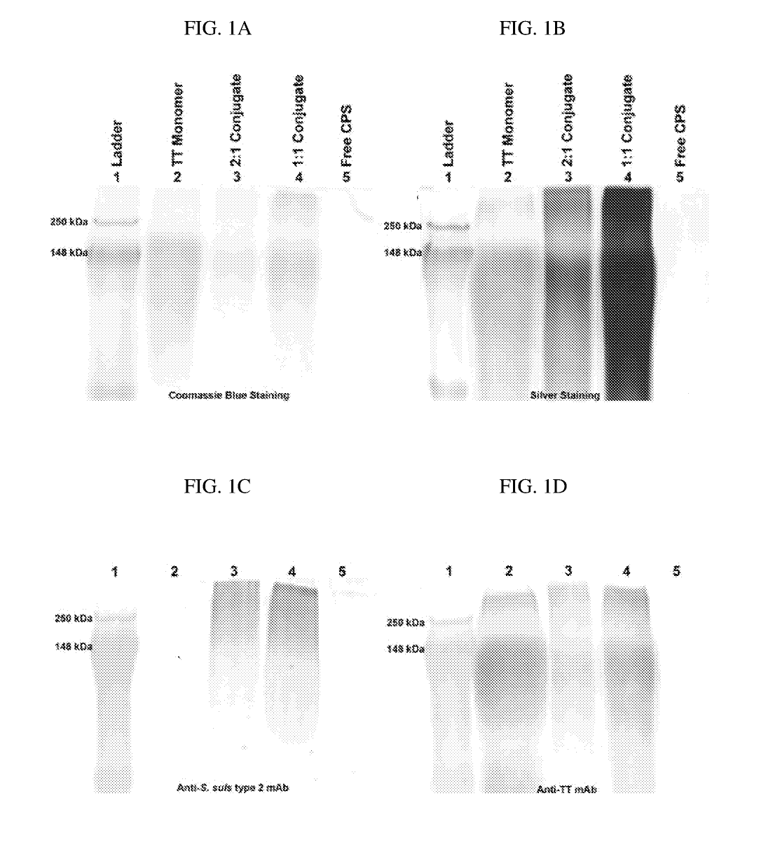

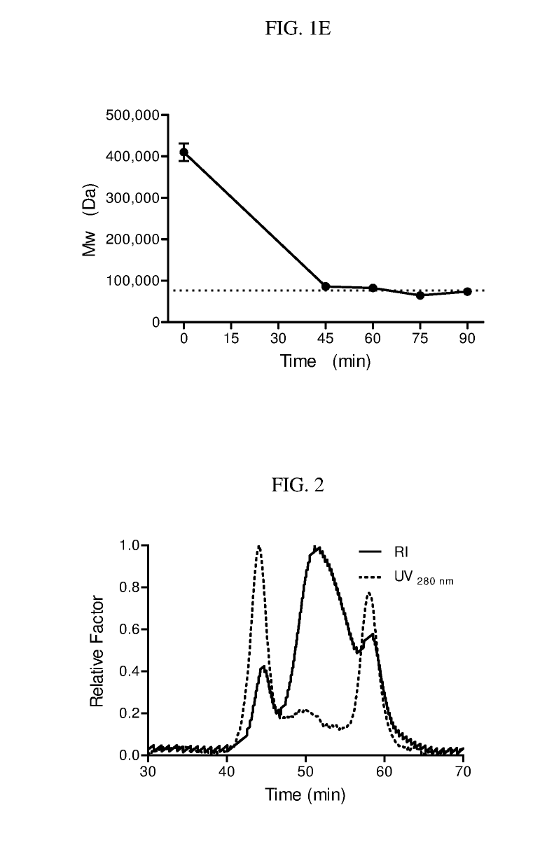

The optimal conditions for sonication were determined in pre-tests using different time points. By monitoring CPS M_{w} of samples by SEC-MALS during pre-tests, it was shown that depolymerization plateaued after 45 min of sonication (FIG. 1E). Based on these observations, we selected a depolymerization time of 60 min of sonication at which two different lots were produced with reproducible results giving an average M_{w} of 115,000 Da (113,000-118,000 Da; Table 1). ¹H NMR investigations of these two lots found no structural alteration of the polysaccharide other than the depolymerization itself (data not shown). These depolymerized CPSs were used in the subsequent preparation of the conjugate vaccine formulations.

TABLE 1
Size-exclusion chromatography coupled with multi-angle light scattering
(SEC-MALS) data for the depolymerized polysaccharide lots.

| | Depolymerized CPS^{a} | | |
|---|---|---|---|
| | M_{w} (g/mol) | R_{z} (nm) | M_{w}/Mₙ |
| Lot I | 1.128 × 10⁵ (0.02%) | 14.0 (0.2%) | 1.003 (0.02%) |
| Lot II | 1.180 × 10⁵ (0.05%) | 17.6 (0.3%) | 1.001 (0.07%) |

^{a}Depolymerized polysaccharide was obtained by ultrasonic irradiation.
Note:
M_{w}, weight-average molar mass; R_{z}, z-average radius of gyration; M_{w}/Mₙ, polydispersity. Values in parentheses represent relative standard deviations.

### 2. Mild Periodate Oxidation of Depolymerized CPS:

The presence of sialic acid (Neu5Ac) as a constituent in the repeating unit sequence of S. suis serotype 2 CPS granted the use of mild conditions in order to achieve an oxidative cleavage between C8-C9 of the glycerol side-chain, thus leaving free terminal aldehydes as reacting groups for subsequent conjugation to TT by reductive amination (Reuter et al. Glycoconj J 1989; 6:35-44). The desired percentage of oxidation is also a critical parameter: too little reactive groups will yield a poor conjugate while too many will leave few intact epitopes of the native polysaccharide (Reuter et al, 1989). To preserve CPS immunogenicity, a 10% level of oxidation was targeted, leaving 90% of all Neu5Ac untouched. For a ˜115,000 Da long CPS, this resulted in an average of 9 oxidized Neu5Ac per chain. Following pre-tests, 0.1 equivalent of sodium periodate per Neu5Ac was selected, and the two different CPS lots were oxidized. Reproducible oxidation levels at C8 of 9.2-9.4% were obtained as determined by GC-FID analysis of the peracetylated methyl glycosides. No oxidation at C7 was observed under these conditions. ¹H NMR investigations found no other structural modification of the polysaccharide (data not shown).

Depolymerized S. suis serotype 2 CPS (8.8 mg, 6.7 μmol) was incubated with 620 μM of sodium periodate in 1.1 ml of water, in the dark, with a stirring magnet at room temperature for 1 h. An excess of two equivalents of triethylene glycol per periodate was added for 1 h to consume any residual periodate. The mixture was dialyzed against water (Spectra/Por, MWCO 1,000; Spectrum Laboratories) and lyophilized. The optimal conditions for oxidation were determined in preliminary tests (data not shown).

The degree of oxidation of the sialic acid (Neu5Ac) residues was assessed by gas chromatography (GC) analysis of the peracetylated methyl glycosides adapted from a previously described method (Houde et al. Infect Immun. 2012; 80:506-17). Briefly, oxidized CPS (0.4 mg) was reduced by adding 100 μl of NaBH₄ (10 mg/ml) in water for 1 h at room temperature. The reaction was quenched with 5% acetic acid solution in methanol and evaporated to dryness using a stream of N₂. Evaporations were repeated 3 times by the addition of 250 μl of methanol each time. The composition of the residue was determined by methanolysis. To this aim, methanol (465 μl) and acetyl chloride (35 μl), which generate HCl, were added to the residue. The solution was heated for 17 h at 75° C., evaporated to dryness, followed by addition of 500 μl of tert-butanol and evaporated to dryness again. The methyl glycosides were acetylated with 150 μl of pyridine and 150 μl of acetic anhydride at 100° C. for 20 min. The cooled solution was partitioned with 5 ml of water and 1 ml of CH₂Cl₂. The organic layer containing the peracetylated methyl glycosides was analyzed by GC using flame ionization detection (GC-FID). GC-FID analysis was done on a Hewlett-Packard model 7890 gas chromatograph equipped with a 30-m by 0.32-mm (0.25-μm particle size) HP-5 capillary column (Agilent Technologies, Santa Clara, Calif., USA) using the following temperature program: 50° C. for 2 min, an increase of 30° C./min to 150° C., then an increase of 3° C./min to 230° C., and a hold for 5 min. The temperatures of the injector and the flame ionization detector were 225° C. and 250° C., respectively.

### 3. Purification of Tetanus Toxoid (TT) Monomer:

TT monomer was obtained by gel filtration chromatography before conjugation. One milliliter of a liquid preparation containing 4.5 mg/ml protein (as determined by the modified Lowry protein assay) was loaded onto a XK16-100 column filled with Superdex 200 Prep Grade (GE Healthcare Life Sciences, Uppsala, Sweden) equilibrated in PBS (20 mM NaHPO₄ pH 7.2, 150 mM NaCl) and eluted with the same buffer. The protein eluted from the column in two peaks: the earlier eluting peak contained oligomerized toxoid, and the later eluting peak, corresponding to a Mᵣ of 150,000, contained tetanus toxoid monomer. Fractions corresponding to the later (monomer) peak were pooled, desalted against deionized water and concentrated using Centricon Plus-70 centrifugal filter device (30K Ultracel PL membrane; Millipore, Billerica, Mass., USA), then lyophilized.

### 4. Conjugation of Type 2 CPS to TT by Reductive Amination:

Periodate treated type 2 CPS (3.6 mg, 40 nmol) and purified TT monomer (3.0 mg, 20 nmol) were dissolved in 2.2 ml of 0.1 M sodium bicarbonate pH 8.1 for the 2:1 conjugate ratio. Sodium cyanoborohydride (7.5 mg, 120 μmol) was added, and the mixture was incubated at 37° C. with orbital agitation for 2 days. For the 1:1 conjugate ratio, conjugation was performed in the same manner as described above except by using 1.8 mg (20 nmol) of oxidized CPS. Sodium borohydride (4.7 mg, 124 μmol) was then added to the reaction mixture to reduce any remaining free aldehyde groups. Conjugate preparations were extensively dialyzed against water (Spectra/Por, MWCO 3,500; Spectrum Laboratories) and lyophilized. Conjugation was controlled by Gel Shift on SDS-PAGE, immunoblotting and high-performance liquid chromatography (HPLC) as described below. The conditions for conjugation by reductive amination were determined in pre-tests using different CPS to TT ratios, different % of CPS oxidation and different incubation times (data not shown).

The depolymerized-oxidized CPS and purified TT monomer were conjugated at a molar ratio of 2 chains of CPS:1 TT or at a molar ratio of 1:1 by reductive amination (Wessels et al. J Clin Invest 1990; 86:1428-33). The optimal incubation time for conjugation was found to be 2 days during pre-tests (data not shown). After incubation, remaining aldehyde groups were reduced by the addition of sodium borohydride. Reagents were then eliminated from the conjugate mixes by extensive dialysis against water.

Two conjugate vaccine formulations were obtained with different CPS:TT ratios. The 2:1 conjugate vaccine was found to be the most immunogenic, namely resulting in significantly higher titers of IgG2b and IgG2c anti-CPS isotypes. This difference in immunogenicity may arise from the higher percentage of total CPS in the 2:1 than in the 1:1 conjugate vaccine (55% and 37%, respectively), which might influence the capacity of the conjugate to modulate the immune cells, including antigen-presenting cells (APCs), presumably through its higher molecular weight/size that might ease uptake and internalization.

### Example 2

### Conjugate Detection & Purification

Gel Shift, Western Blot and HPLC Analysis Confirmed Successful Conjugation of CPS to TT:

The presence of conjugates in the different preparations was verified by Gel shift and Western blot experiments (FIGS. 1A-1D) and by HPLC analysis (FIG. 2). For the Gel shift experiments, both Coomassie Blue (FIG. 1A) and Silver staining (FIG. 1B) showed a considerable shift from the purified TT monomer at 150 kDa (lane 2) to a thick band of over 250 kDa in the conjugates (lanes 3-4). This shift resulted from the covalent addition of a random number of 115 kDa CPS chains to the protein. Neither Coomassie Blue (FIG. 1A), Silver staining (FIG. 1B) nor Western Blot using an anti-CPS mAb (FIG. 1C) revealed any band for the depolymerized CPS included as a control in all gels (lane 5), illustrating its weak capacity to migrate through the gels under these assay conditions. Control staining using an anti-TT mAb (FIG. 1D) shows that the epitope to which the monoclonal antibody binds was preserved in the conjugates. Preservation of TT antigenicity is essential since it is the key mechanism allowing for the production of a T cell-dependent anti-CPS humoral response. It should be noted that differences in signal intensities between the 2:1 and 1:1 conjugate preparations (FIGS. 1A-1D, lanes 3-4) are likely related to the total amounts of protein content (4.5 μg vs. 6.3 μg, respectively) within the 10 μg loaded sample per lane. Positive signals were only observed for the bands greater than 250 kDa when using an anti-CPS mAb indicating the covalent nature of the linkage between CPS and TT in the conjugates (FIG. 1C, lanes 3-4).

HPLC analysis (FIG. 2) showed the elution of the conjugate (>250 kDa), elution of free CPS (100 kDa) and of free TT (150 kDa). By integrating UV_{280 nm} signal from the chromatograms, it was estimated that 48±6% (mean±SD) of the protein content from the mixture is indeed found in the conjugate fraction. Taken together, Gel shift and Western blot experiments combined with HPLC analysis of the two conjugate samples revealed the presence of conjugates in the 2 CPS:1 TT and 1:1 preparations.

### Example 3

### Mouse Immunization:

Five to 6 week-old C57BL/6 female mice (Charles River, Wilmington, Mass., USA) were immunized subcutaneously with different doses of the S. suis conjugate preparations in 0.1 ml PBS on day 0 and boosted on day 21. In a first set of experiments aimed to compare different adjuvants, 3 groups (n=10) received 25 μg of the 2:1 conjugate vaccine formulation dissolved in PBS adjuvanted with either 20 μg of CpG oligodeoxyribonucleotide (ODN) 1826 (InvivoGen, San Diego, Calif., USA), STIMUNE® (Prionics, La Vista, Nebr., USA) or TITERMAX® Gold (CytRx Corporation, Norcross, Ga., USA) following manufacturer's recommendations. Three placebo groups (n=5) received only PBS adjuvanted as described above. In a second set of experiments, a dose-response study was performed using groups of mice (n=8) immunized with either 1, 2.5, 5 or 25 μg of the 2:1 conjugate vaccine emulsified 1:1 (v/v) with TITERMAX® Gold. Mice (n=8) immunized with similar doses of free (unconjugated) depolymerized CPS emulsified with TITERMAX® Gold were included for comparison purposes. A placebo group (n=5) was also included. In a third set of experiments, to compare the efficiency of different conjugates, groups of mice (n=10) received either 25 μg of the 1:1 conjugate vaccine formulation, HPLC-purified conjugate fraction or a free (unconjugated) mixture of 2 CPS: 1 TT. All preparations were emulsified with TITERMAX® Gold and a placebo group was also included.

In all experiments, to follow antibody responses, mice were bled (10 μl) weekly on days −1, 7, 14, 21, 28, 35, and 41 post-immunization by the tail vein. Diluted blood was directly used in the ELISA test as described above. At day 42 post-immunization mice were humanely euthanized and sera collected and frozen at −80° C. for ELISA Ig titration and isotyping, and for OPA analyses (as described above).

Emulsifying Adjuvants Present Higher Immunomodulatory Properties than CpG ODN for a Polysaccharide Antigen:

Using the 2:1 conjugate formulation, optimization of the immunization protocol was performed in a murine model using inbred C57BL/6 mice.

During pre-trials, it was observed that conjugation alone was not enough to induce a robust immunological response against S. suis type 2 CPS (data not shown). In this regard, subunit vaccines are known to induce more potent and durable antigen-specific immunity if combined with an adjuvant (O'Hagan and Valiante. Nat Rev Drug Discov. 2003; 2:727-35). It has been shown that adjuvants can not only improve the immunogenicity of conjugate vaccines but also to differently direct the anti-polysaccharide antibody isotype switch towards the desired IgG subclasses (Chu et al. Infect Immun. 2000; 68:1450-6; and Fattom et al. Vaccine. 1995; 13: 1288-93). CpG ODN 1826, currently undergoing clinical trials for use in human vaccines (Bode et al. Expert Rev Vaccines 2011; 10: 499-511), was shown to enhance the isotype switching from IgM to IgG2a and IgG3 subclasses for pneumococcal conjugates in a serotype- and mouse age-dependent manner (Chu et al. J Exp Med. 1997; 186: 1623-31; Chu et al. Infect Immun. 2000; 68:1450-6; and Kovarik et al. Immunology. 2001; 102: 67-76).

The performance of three different adjuvants was compared. CpG ODN is a synthetic version of bacterial oligonucleotide with unmethylated CpG motifs and acts as a Toll-like receptor 9 (TLR9) ligand with immunostimulatory properties toward a Th1 response (Chu et al., 1997). STIMUNE® (Specol) is a water-in-oil adjuvant composed of purified and defined mineral oil (Markol 52) with Span 85 and Tween 85 as emulsifiers (Stills H F. ILAR Journal 2005; 46:280-93). It has been used as a good alternative to Freund's adjuvant for weak immunogens in animals, such as mice and pigs (Leenaars et al. Vet Immunol Immunopathol. 1994; 40:225-41). TITERMAX® Gold is also a water-in-oil adjuvant consisting of squalene as a metabolizable oil, sorbitan monooleate 80 as an emulsifier and CRL8300 (a patented block copolymer) and microparticulate silica as stabilizers (Stills, 2005). TITERMAX® Gold has been suggested as a superior alternative to Freund's adjuvant providing comparable titers with fewer injections and less undesired reactivity in mice (Bennett et al. J Immunol Methods. 1992; 153:31-40).

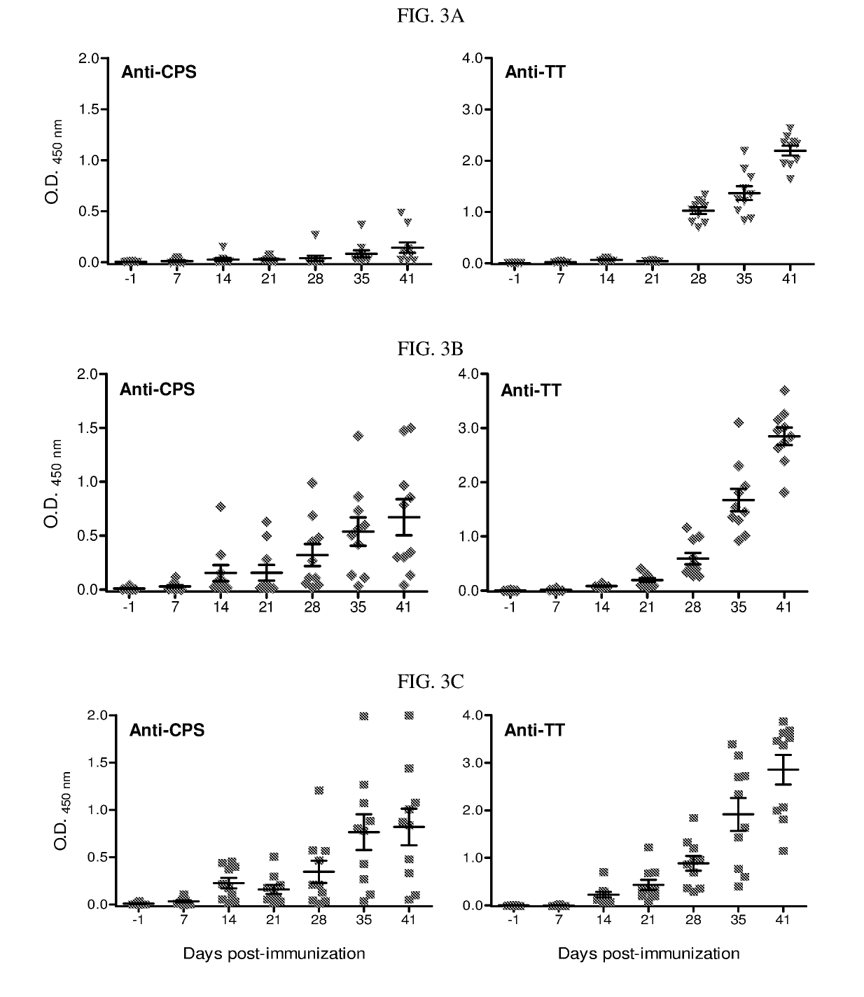

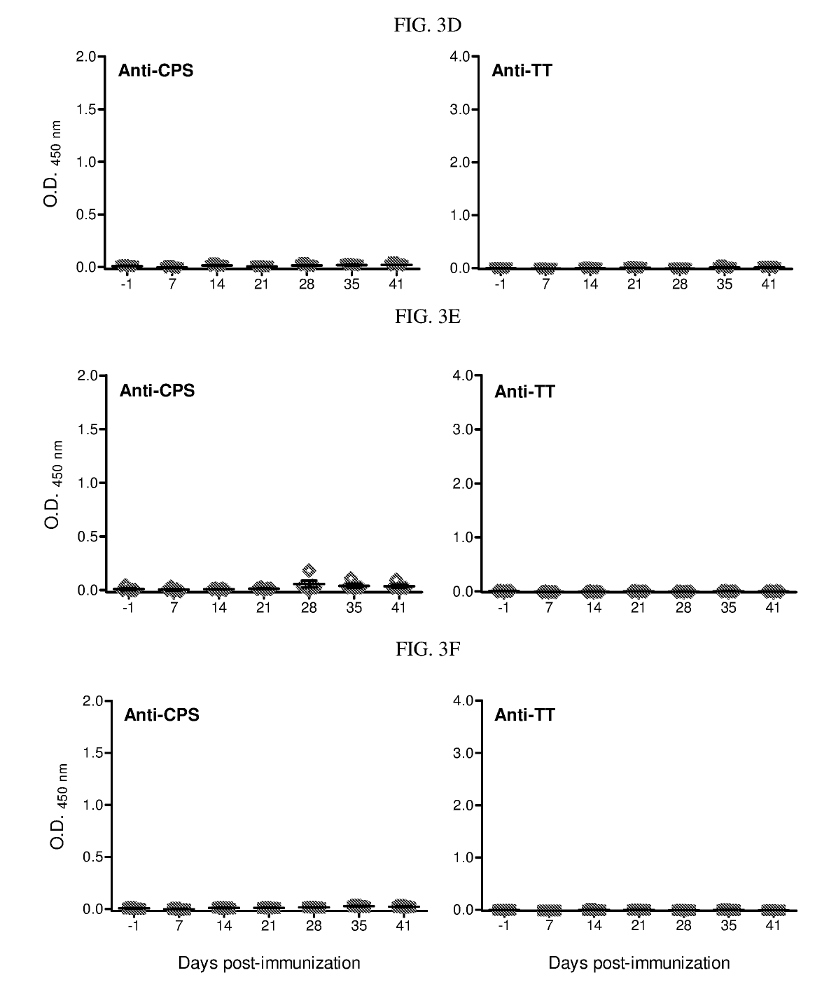

Based on the literature (Sommariva et al. J Transl Med. 2013; 11:25), a dose of 20 μg of CpG was chosen to be added for adjuvanting. In parallel, conjugates were emulsified with recommended ratios of 4 parts aqueous antigen per 5 parts adjuvant for STIMUNE®, or 1:1 for TITERMAX® Gold. Doses of 25 μg of the 2:1 conjugate vaccine for each adjuvant were administered to mice on days 0 and 21. The kinetics of total Ig[G+M] antibody responses against CPS or TT were followed weekly from tail vein blood samples (FIG. 3A-C). Overall, CpG ODN 1826 (FIG. 3A) gave the lowest anti-CPS and anti-TT responses. Also, anti-CPS Ig isotyping showed a strict IgM isotype response (data not shown). In contrast, STIMUNE® (FIG. 3B) and TITERMAX® Gold (FIG. 3C) gave comparable strong anti-CPS and anti-TT total Ig[G+M] responses. Furthermore, anti-CPS Ig isotype switching was observed with both emulsifying adjuvants (see below). Albeit a memory antibody response against TT was observed with all three adjuvants, STIMUNE® and TITERMAX® Gold induced faster and higher anti-CPS antibody levels after boost, suggesting that generation of immunological memory against the CPS antigen is favored by these emulsifying adjuvants. Finally, it should be noted that all placebo mice, injected only with PBS and adjuvant, did not produce any non-specific antibody response (FIG. 3D-F).

As TITERMAX® Gold is recognized as one of the best adjuvants for mice (Jennings. ILAR Journal. 1995; 37:119-25; and Kateregga et al. BMC Vet Res. 2012; 8:63), it was selected for further immunizations with this species.

Dose-Response Effect on Antibody Levels is Observed with the Conjugate Vaccine:

Using TITERMAX® Gold as the adjuvant, mice were immunized on days 0 and 21 with doses of 1, 2.5, 5 or 25 μg of the 2:1 conjugate vaccine to evaluate the dose-response effect on antibody production. Groups of mice were also immunized with different doses of S. suis serotype 2 free (unconjugated) CPS to assess if it could be immunogenic by itself when adjuvanted with TITERMAX® Gold.

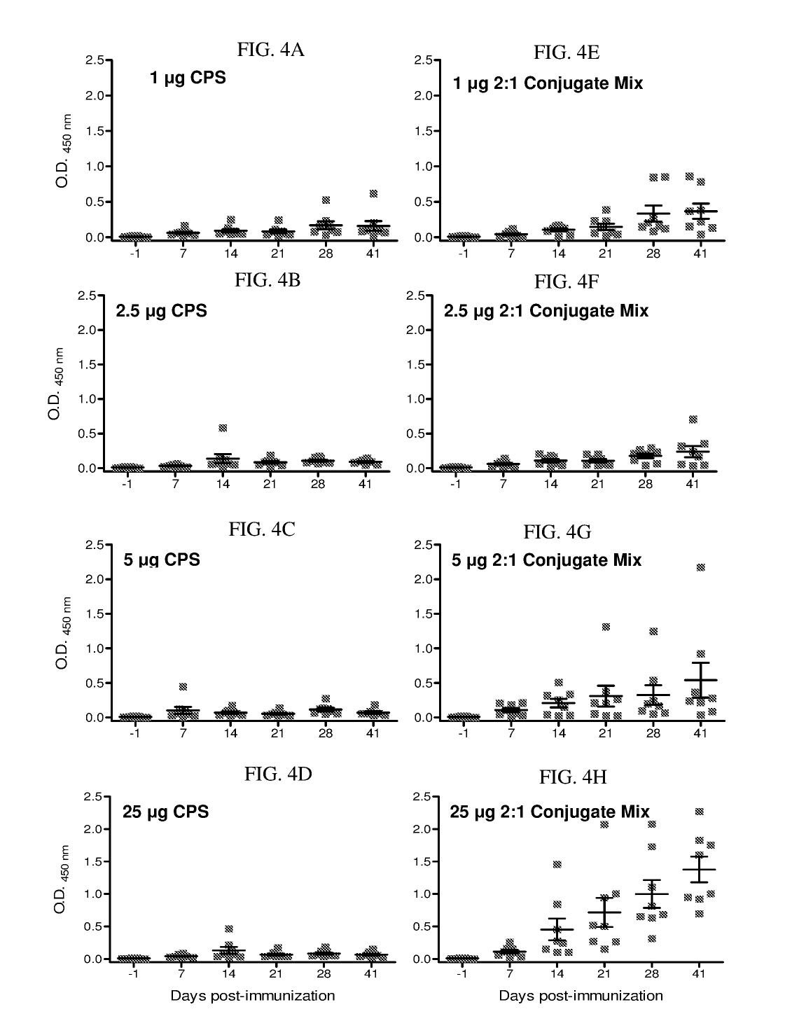

Even at a high dose (25 μg) of free CPS, no significant total Ig[G+M] primary or memory antibody responses were observed throughout the immunization period (FIGS. 4A-4H). In contrast, a dose-response effect was observed when mice were immunized with the 2:1 conjugate preparation, with the 25 μg dose yielding the highest total Ig[G+M] anti-CPS antibody response as measured on weekly collected blood samples.

Conjugation of S. suis Type 2 CPS to TT Induces Antibody Isotype Switching in Mice:

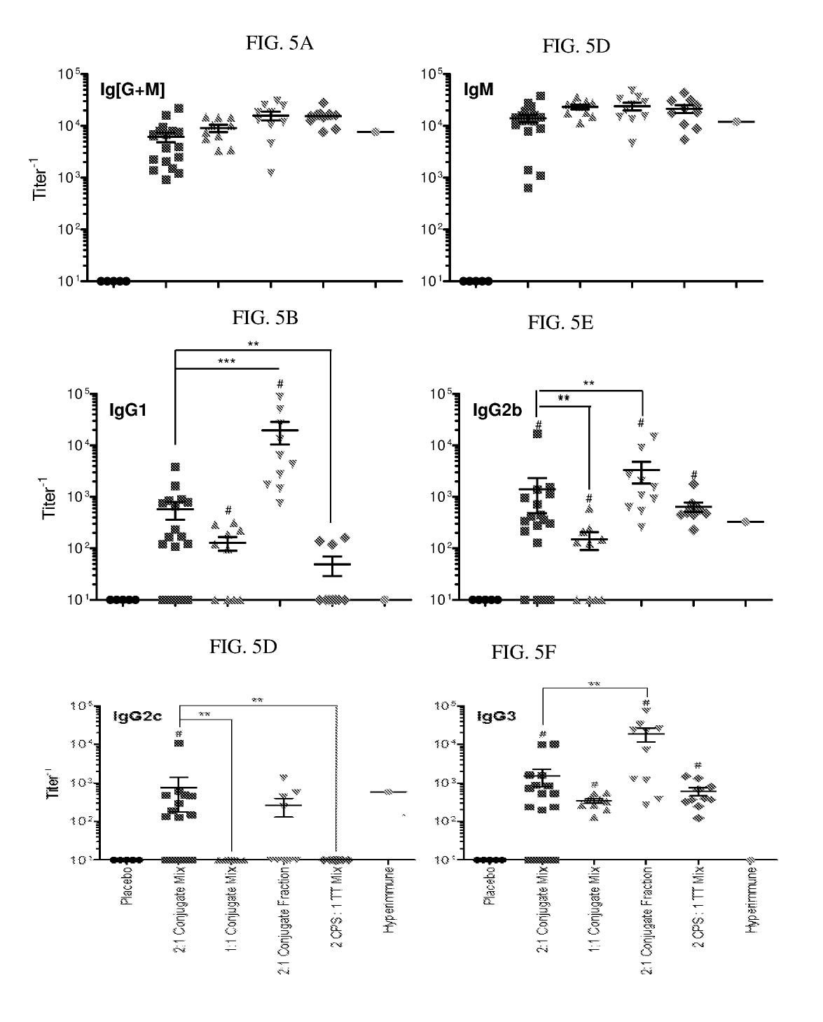

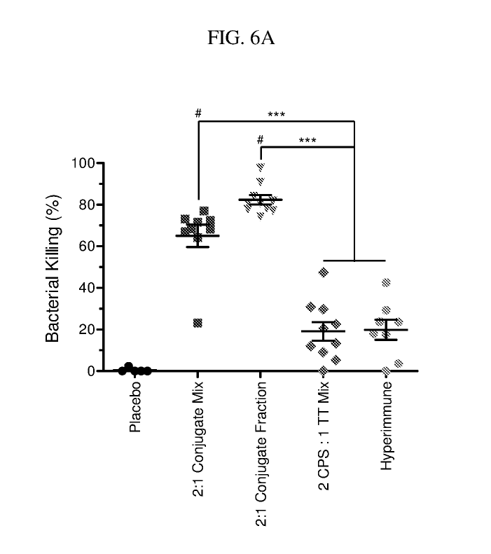

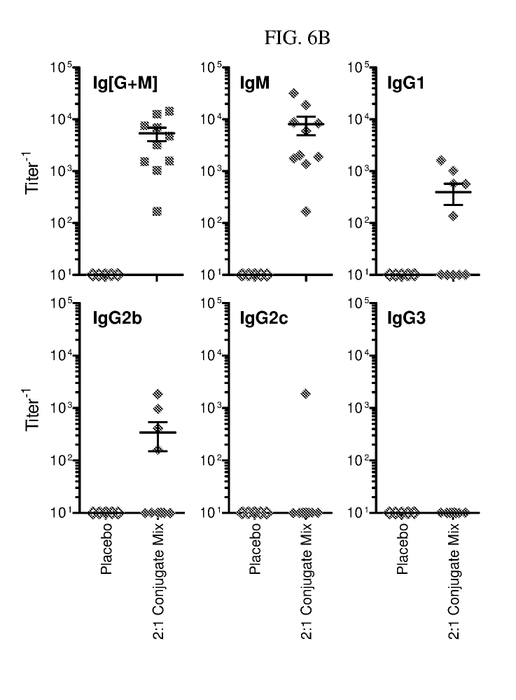

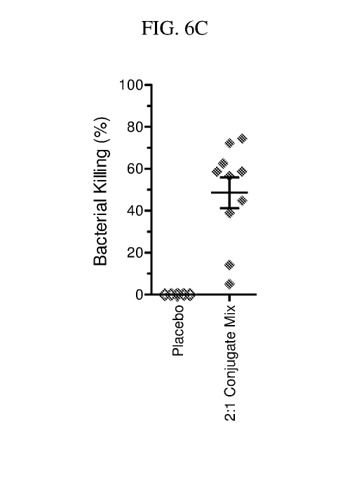

Not only a stronger response following boost (as illustrated in FIGS. 3A-3F and 4A-4H), but also antibody isotype switching are good indicators of conjugate immunogenicity and ability to induce a T cell-dependent response. As such, titers of the different anti-CPS antibody isotypes were determined in mice immunized with 25 μg of the 2:1 conjugate vaccine adjuvanted with TITERMAX® Gold. As shown in FIGS. 5A-5F, not only strong IgM titers, but also high levels of all IgG subclasses were observed, including IgG1, IgG2b, IgG2c and IgG3 specific for the CPS antigen. To evaluate if isotype switching was dependent on the adjuvant, serum samples of mice immunized with 25 μg of the 2:1 conjugate preparation adjuvanted with STIMUNE® were analyzed. STIMUNE® also induced isotype switching in mice; however, levels were lower and profiles differed from those observed with TITERMAX® Gold, with no production of the IgG2c and IgG3 subclasses (FIG. 6B).

To determine the effect of CPS to TT ratio on the conjugate immunogenicity, another conjugate formulation, this time using a ratio of 1 CPS: 1 TT was prepared (displayed in FIG. 1 A-D) and emulsified in TITERMAX® Gold.

Immunized mice showed similar IgM titers, reduced (but not significantly different) IgG1 and IgG3 titers, and significantly lower IgG2b titers (P<0.01) than those induced by the 2:1 conjugate formulation in TITERMAX® Gold (FIG. 5 A-F). Interestingly, the 1:1 conjugate vaccine failed to induce significant titers of the IgG2c subclass.

To demonstrate that the observed immunogenicity was in fact due to the conjugate present in the vaccine formulation, and not only due to remaining free CPS and TT, two additional controls were included in the study. A first control was the HPLC-isolated specific fraction corresponding to the conjugate from the 2:1 conjugate preparation. The second control was a mixture of free CPS and free TT in the same ratio of 2:1 as before conjugation. In general no major differences were observed between 2:1 conjugate formulation and the specific 2:1 conjugate HPLC-fraction, yet, higher titers of IgG1, IgG2b, and IgG3 were observed with the later preparation (FIGS. 5 B, E, and F, P<0.01). In contrast, the mixture of unconjugated CPS and TT gave a strong IgM titer but very low titers of IgG1 (FIGS. 5B and D) compared to the 2:1 conjugate formulation (P<0.01). In addition, no production of IgG2c (FIG. 5 C) subclass was observed in mice immunized with this control unconjugated preparation.

Finally, the control hyperimmune sera from mice repeatedly injected with heat-killed bacteria resulted in a high titer of IgM (FIG. 5D), production of IgG2b (FIG. 5E), and IgG2c (FIG. 5C), but absence of IgG1 (FIG. 5B), and IgG3 (FIG. 5F), subclasses against the CPS antigen (FIG. 5 A-F). Similar results were obtained when mice were hyperimmunized with heat-killed bacteria adjuvanted in TITERMAX® Gold (data not shown).

### Functional Activity of Antibodies:

A strong antibody response does not necessarily reflect upon the protection of an individual (Goyette-Desjardins et al. Methods Mol Biol. 2015; 1331:81-92). In this regard, functional assays are preferred, like the opsonophagocytosis assay (OPA), a recognized correlate of protective immunity against extracellular encapsulated Gram-positive bacteria, such as S. pneumonia (Plotkin S A. Clin Vaccine Immunol. 2010; 17:1055-65; Song J Y, et al. J Infect Chemother. 2013; 19:412-25; and Romero-Steiner et al. Clin Vaccine Immunol. 2006; 13:165-9). During the OPA, opsonizing antibodies from the immunized serum will opsonize the target bacteria, which in turn triggers activation of the classical pathway of the complement. Both deposited antibodies and/or complement will be recognized by Fc receptors and complement receptors, respectively, triggering an enhanced immune response by blood leukocytes which results in bacterial phagocytosis and bactericidal activity (Goyette-Desjardins et al. Methods Mol Biol. 2015; 1331:81-92; Guilliams et al. Nat Rev Immunol. 2014; 14: 94-108; Underhill and Ozinsky. Annu Rev Immunol. 2002; 20:825-52; and Ricklin et al. Nat Immunol. 2010; 11:785-97). Specific cell type activation depends on the Ig isotypes/subclasses present in the immune serum, since each isotype/subclass possesses different binding preferences to Fc receptors, which differently influences the cell response (Goyette-Desjardins et al., Methods Mol Biol. 2015; 1331:81-92; and Underhill and Ozinsky, 2002). Besides IgM, the predominant subclass of protective antibodies to TI antigens in mice is the IgG3 (Lee et al. Crit Rev Microbiol. 2003; 29:333-49; Perlmutter et al. J Immunol. 1978; 121:566-72; Rubinstein and Stein. J Immunol. 1988; 141:4352-6; and Schreiber et al. J Infect Dis. 1993; 167:221-6). A study using mouse monoclonal antibodies proposed that the type 1 subclasses (IgG3>>IgG2b≥IgG2a) are superior in both opsonophagocytosis activity and complement activation than the type 2 IgG1 subclass. Yet, these functional properties of mouse IgG subclasses seem to depend on the target antigen (protein vs. carbohydrate), antigen distribution, and the susceptibility of the bacteria for antibody/complement attack (Michaelsen et al. Scand J Immunol. 2004; 59:34-9; and McLay et al. J Immunol. 2002; 168:3437-43).

Instead of using a cell line or a single cell type, the OPA was standardized using whole blood from naïve mice (Goyette-Desjardins et al., Methods Mol Biol. 2015; 1331:81-92). This model takes into account all blood leukocytes and thus represents a more realistic model of the complex interactions between all immune cells and the bacteria during a systemic infection, as is the case for S. suis.

As shown in FIG. 6A, sera from mice immunized with the 2:1 conjugate vaccine adjuvanted with TITERMAX® Gold induced high bacterial killing levels ranging from 64-77%. Sera from mice immunized with the 2:1 conjugate HPLC-fraction gave higher, but not significantly different, bacterial killing values ranging from 74-98% (FIG. 6A). The effect of the adjuvant was also evaluated in the OPA test; sera from mice immunized with the 2:1 conjugate vaccine adjuvanted with STIMUNE® induced bacterial killing levels ranging from 39-74% (FIG. 6C), which were not significantly different from those induced by TITERMAX® Gold (P>0.05). When the OPA was performed using sera from mice immunized with unconjugated CPS and TT mixture, significant lower values (between 0-47%) of bacterial killing were observed compared to conjugates (FIG. 6A; P<0.001). Pooled sera from hyperimmunized mice gave bacterial killing values highly similar to those of the unconjugated mixture (FIG. 6A; P>0.05).

Thus, the results demonstrated that the two groups which obtained the highest bacterial killing values were the 2:1 conjugate vaccine and the 2:1 conjugate HPLC-fraction adjuvanted with TITERMAX® Gold, both containing the highest titers of type 1 IgG subclasses, namely IgG3, IgG2b and IgG2c. They were closely followed by the 2:1 conjugate vaccine adjuvanted with STIMUNE® producing appreciable titers of IgG2b, although this group lacks production of IgG3 and IgG2c. In contrast, control mouse groups immunized with the mixture of free CPS and free TT or mice hyperimmunized with killed-bacteria failed to adequately perform in the OPA test, probably due to the combined absence or low levels of several IgG subclasses, including IgG1.

### Example 4

### Immunogenicity and Protection in Pigs:

Based on previous results, the 2:1 conjugate formulation was selected to evaluate the immunogenicity and protection in the S. suis natural host: the pig. The adjuvant STIMUNE® was chosen as it had been previously included in S. suis bacterin-based vaccines (Wisselink et al. Vet Microbiol. 2002; 84:155-68; and Swildens et al. Vet Rec. 2007; 160:619-21). The performance of the conjugate was compared to that of a S. suis type 2 bacterin adjuvanted with STIMUNE®.

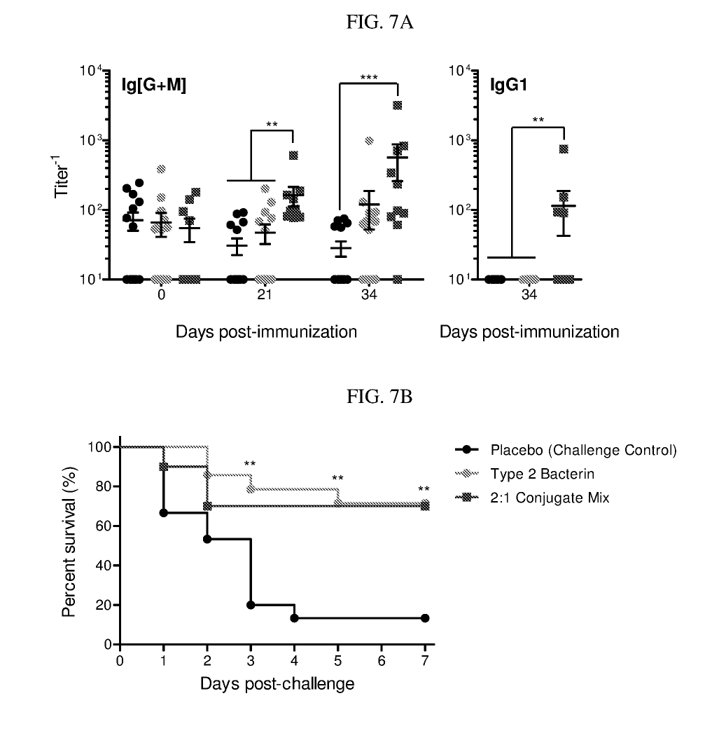

Pigs were injected intramuscularly twice at a 3-week interval and serum samples were collected on days 0, 21, and 34 for titration and isotyping of anti-CPS antibodies (FIG. 7A). On day 21 post-immunization, total Ig[G+M] anti-CPS titers induced by the 2:1 conjugate vaccine were significantly higher (P<0.01) than those of the placebo and bacterin. After boosting, on day 34 post-immunization, an increase in anti-CPS titers was observed for both vaccinated groups compared to day 21. However, only the titers from pigs vaccinated with the 2:1 conjugate formulation were significantly higher than the placebo control group (P<0.001). Titers of the different swine IgG subclasses, namely IgG1 and IgG2, were also assayed post-boost injection (day 34). Forty percent of pigs immunized with the 2:1 conjugate vaccine showed significant levels of anti-CPS IgG1 subclass (FIG. 7A). No switch to the IgG2 subclass was observed (data not shown). In contrast, vaccination of pigs with the bacterin failed to induce anti-CPS Ig class switch (FIG. 7A and data not shown).

On study day 36, pigs were challenged intraperitoneally with a dose of 3×10⁹ CFU of ATCC 700794, a virulent S. suis serotype 2 strain. Most pigs in the placebo group died during the systemic phase of S. suis infection, reaching a mortality of 86.7%. In contrast, pigs immunized with the bacterin or the 2:1 conjugate vaccine both showed mortality of 28.6% and of 30.0%, respectively. Analysis of survival curves (FIG. 7B) showed a significant difference as soon as day 3 between both immunized groups and the placebo group (P=0.009). Protection induced by the 2:1 conjugate vaccine was similar to that of the control type 2 bacterin during the systemic phase of a S. suis challenge infection in pigs.

Pigs were also monitored for clinical signs (behavior, locomotion problems or CNS signs) for seven consecutive days after challenge. In 31.6% of all observations for the bacterin-vaccinated group and in 28.1% of all observations for the conjugate-vaccinated group abnormal behavior was observed. This was significantly lower compared to the findings in the placebo group, in which 90.7% of the observations revealed abnormal behavior (Table 2, adjusted P value <0.05). Lameness was observed in 26.3% of all observations for the bacterin-vaccinated group and in 33.5% of all observations for the conjugate-vaccinated group compared to 89.3% for the placebo group (Table 2, adjusted P value <0.05). These differences were also observed when the distribution of clinical scores was analyzed daily for each group (data not shown). CNS signs were observed only in few pigs and no statistically significant differences were observed between the three challenged groups (Table 2). This can be explained by the fact that animals were observed for only a 7 day-period post-challenge, as the study design mainly focused on the systemic phase of the disease.

TABLE 2
Clinical evaluation of immunized pigs after experimental
challenge with S. suis serotype 2^{a}.

| | Abnormal | | Abnormal | | CNS clinical | |
|---|---|---|---|---|---|---|
| | behavior | | locomotion | | signs | |
| Groups | %^{b} | P value^{c} | %^{b} | P value^{c} | %^{b} | P value^{c} |
| Type 2 Bacterin | 31.6 | 0.0209 | 26.3 | 0.0064 | 1.2 | NS |
| 2:1 Conjugate | 28.1 | 0.0308 | 33.5 | 0.0335 | 0 | NS |
| Placebo | 90.7 | — | 89.3 | — | 2.6 | — |
| (challenge control) | | | | | | |

^{a}Assessed were behavior, including any behavior indicating an effect of challenge on the central nervous system (CNS) and locomotion. The observations for behavior were numerically scored as follows: 0 = physiological, 1 = depression, 2 = apathy. Observations for locomotion were scored as 0 = physiological, 1 = slightly to moderately lame, 2 = severely lame/reluctant to stand, 3 = animal partially/completely down; animals can rise, but lie down again within 10 seconds. CNS signs were scored as 0 = absent and 1 = present.
^{b}Assessment of cumulative observation period. Percent of evaluations where behavior, locomotion or CNS signs gave a value >0 across days. Data are expressed as least squares means (back-transformed, %).
^{c}Adjusted P-value (Scheffé's test): all values compared to challenge control group; strict control group not included in assessment.
NS, non-significant with P ≥ 0.05.

All pigs found dead as well as all euthanized pigs were necropsied. The frequency of gross lesions in the thoracic cavity (i.e. fibrin, excess fluid, pericarditis) or in the joints was overall reduced in vaccinated animals compared to the placebo group. Yet observed differences did not reach statistical significance (Table 3). The conjugate vaccine significantly reduced the challenge strain recovery from joint swabs (P<0.01). The S. suis challenge strain was also less frequently isolated from the meningeal and pericardial swabs compared to the placebo group, yet differences were not statistically different (Table 4).

TABLE 3
Gross pathology observations from necropsy (or
post-mortem examination) of challenged pigs^{a}.

| | Thoracic Cavity | | Joint | |
|---|---|---|---|---|
| Groups | %^{b} | P value^{c} | %^{b} | P value^{c} |
| Type 2 Bacterin | 35.7 | 0.0294 | 35.7 | NS |
| 2:1 Conjugate | 40.0 | NS | 30.0 | NS |
| Placebo (challenge control) | 80.0 | — | 53.3 | — |

^{a}Signs of inflammation of the thoracic cavity (including serosal surfaces, heart and lung) and the joints (including excess fluid, fibrin, swelling) were recorded.
^{b}Percentage of animals with pathological findings/observations.
^{c}P-value: all values compared to challenge control group; strict control group not included in assessment.
NS, non-significant with P ≥ 0.05.

TABLE 4
Streptococcus suis serotype 2 recovery from swabs at necropsy
(or post-mortem examination) of challenged pigs^{a}.

| | Meninges | | Pericard | | Joints | |
|---|---|---|---|---|---|---|
| Groups | %^{b} | P value^{c} | %^{b} | P value^{c} | %^{b} | P value^{c} |
| Type 2 Bacterin | 21.4 | 0.0092 | 14.3 | 0.0078 | 14.3 | 0.0007 |
| 2:1 Conjugate | 40.0 | NS | 30.0 | NS | 20.0 | 0.0051 |
| Placebo | 80.0 | — | 66.7 | — | 80.0 | — |
| (challenge control) | | | | | | |

^{a}Culture from swabs were confirmed by morphology, serotyping with type 2 antisera and by S. suis type 2 PCR.
^{b}Percentage of animals with at least one positive S. suis type 2 isolate from swab cultures.
^{c}P-value: all values compared to challenge control group; strict control group not included in assessment.
NS, non-significant with P ≥ 0.05.

Thus, in summary, anti-CPS IgM and IgG1 antibodies were detected and found to be significantly protective in an in vivo lethal-dose challenge with virulent S. suis serotype 2. Although the bacterin induced similar levels of protection than the conjugate vaccine, this protection was not related to anti-CPS antibodies. This is in agreement with previous data showing that whole S. suis (either live or killed) fails to induce significant levels of anti-CPS antibodies in mouse or swine models (Calzas et al. Infect Immun. 2015; 83:441-53). Thus, conjugation of the CPS to a carrier protein is absolutely required to generate opsonizing anti-CPS antibodies, which are known to be highly protective against encapsulated bacteria. On the other hand, protection generated by the bacterin is probably related to anti-protein antibodies. However, and in contrast to CPS, which is a universal antigen for S. suis type 2, protein antigens vary upon the strain origin or sequence type (ST) (Fittipaldi et al. Future Microbiol. 2012; 7:259-79; Galina et al. Can J Vet Res. 1996; 60:72-4; Gottschalk et al. Can J Vet Res. 1998; 62:75-9; Okwumabua et al. FEMS Microbiol Lett. 1999; 181:113-21; Fittipaldi et al. Emerg Infect Dis. 2011; 17: 2239-44; and Li Y, et al. Infect Immun. 2006; 74:305-12) and is therefore not as readily versatile.

Statistical Analyses:

Summaries of and data analyses for the pig study were conducted by a bio-statistician using SAS® Version 9.3. Clinical observations (death, lameness, CNS signs, and behavioral changes) were summarized as frequencies by day and treatment. Incidence of normal versus not normal for each characteristic were analyzed where appropriate using the GLIMMIX procedure of SAS with binomial error and logit link. The model included the fixed effect of treatment and the random effects of litter and residual. In addition, the proportion of the observations for each animal that were not normal was analyzed. Prior to analysis, the proportion was transformed using the arcsine square root transformation. The mixed model included the fixed effect of treatment and the random effects of litter and residual. Comparisons of interest include the following and were evaluated using a two-sided test with alpha=0.05: Groups 1 (bacterin) and 2 (conjugate) vs. 3 (protection provided against challenge with isolate ATCC 700794).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

### REFERENCES

The following references, to the extent that they provide exemplary procedural or other details supplementary to those set forth herein, are specifically incorporated herein by reference.
[1] Gottschalk M. Streptococcosis. In: Zimmerman J J, Ramirez A, Schwartz K J, Stevenson G W, editors. Diseases of swine. 10th ed. Ames, USA: Wiley-Blackwell Publishing; 2012. p. 841-55.
[2] Field H I, Buntain D, Done J T. Studies on piglet mortality. I. Streptococcal meningitis and arthritis. Vet Rec 1954; 66:453-5.
[3] Jansen E J, Dorssen C A. [Meningo-encephalitis in pigs caused by Streptococci]. Tijdschr Diergeneeskd 1951; 76:815-32.
[4] de Moor C E. Septicaemic infections in pigs, caused by haemolytic Streptococci of new Lancefield groups designated R, S and T. Antonie Van Leeuwenhoek 1963; 29:272-80.
[5] Elliott S D. Streptococcal infection in young pigs. I. An immunochemical study of the causative agent (P M Streptococcus). J Hyg (Lond) 1966; 64:205-12.
[6] Windsor R S, Elliott S D. Streptococcal infection in young pigs. I V. An outbreak of streptococcal meningitis in weaned pigs. J Hyg (Lond) 1975; 75:69-78.
[7] Goyette-Desjardins G, Auger J P, Xu J, Segura M, Gottschalk M. Streptococcus suis, an important pig pathogen and emerging zoonotic agent—an update on the worldwide distribution based on serotyping and sequence typing. Emerg Microbes Infect 2014; 3:e45.
[8] Higgins R, Gottschalk M. Streptococcal diseases. In: Straw B E, D'Allaire S, Mengeling W L, Taylor D J, editors. Diseases of swine. 9th ed. Ames, USA: Blackwell Publishing; 2006. p. 769-83.
[9] Sanford S E, Tilker M E. Streptococcus suis type II-associated diseases in swine: observations of a one-year study. J Am Vet Med Assoc 1982; 181:673-6.
[10] Cloutier G, D'Allaire S, Martinez G, Surprenant C, Lacouture S, Gottschalk M. Epidemiology of Streptococcus suis serotype 5 infection in a pig herd with and without clinical disease. Vet Microbiol 2003; 97:135-51.
[11] Segura M. Fisher scientific award lecture—The capsular polysaccharides of Group B Streptococcus and Streptococcus suis differently modulate bacterial interactions with dendritic cells. Can J Microbiol 2012; 58:249-60.
[12] Lapointe L, D'Allaire S, Lebrun A, Lacouture S, Gottschalk M. Antibody response to an autogenous vaccine and serologic profile for Streptococcus suis capsular type 1/2. Can J Vet Res 2002; 66:8-14.
[13] Baums C G, Brüggemann C, Kock C, Beineke A, Waldmann K-H, Valentin-Weigand P. Immunogenicity of an autogenous Streptococcus suis bacterin in preparturient sows and their piglets in relation to protection after weaning. Clin Vaccine Immunol 2010; 17:1589-97.
[14] Wisselink H J, Stockhofe-Zurwieden N, Hilgers L A T, Smith H E. Assessment of protective efficacy of live and killed vaccines based on a non-encapsulated mutant of Streptococcus suis serotype 2. Vet Microbiol 2002; 84:155-68.
[15] Busque P, Higgins R, Caya F, Quessy S. Immunization of pigs against Streptococcus suis serotype 2 infection using a live avirulent strain. Can J Vet Res 1997; 61:275-9.
[16] Fittipaldi N, Harel J, D'Amours B, Lacouture S, Kobisch M, Gottschalk M. Potential use of an unencapsulated and aromatic amino acid-auxotrophic Streptococcus suis mutant as a live attenuated vaccine in swine. Vaccine 2007; 25:3524-35.
[17] Fittipaldi N, Segura M, Grenier D, Gottschalk M. Virulence factors involved in the pathogenesis of the infection caused by the swine pathogen and zoonotic agent Streptococcus suis. Future Microbiol 2012; 7:259-79.
[18] Calzas C, Lemire P, Auray G, Gerdts V, Gottschalk M, Segura M. Antibody response specific to the capsular polysaccharide is impaired in Streptococcus suis serotype 2-infected animals. Infect Immun 2015; 83:441-53.
[19] Song J Y, Moseley M A, Burton R L, Nahm M H. Pneumococcal vaccine and opsonic pneumococcal antibody. J Infect Chemother 2013; 19:412-25.
[20] Heath P T. An update on vaccination against Group B Streptococcus. Expert Rev Vaccines 2011; 10:685-94.
[21] Bottomley M J, Serruto D, Wadi M A P, Klugman K P. Future challenges in the elimination of bacterial meningitis. Vaccine 2012; 30:B78-B86.
[22] Roy R, Shiao T C. Organic chemistry and immunochemical strategies in the design of potent carbohydrate-based vaccines. Chimia 2011; 65:24-9.
[23] Elliott S D, Clifton-Hadley F, Tai J. Streptococcal infection in young pigs. V. An immunogenic polysaccharide from Streptococcus suis type 2 with particular reference to vaccination against streptococcal meningitis in pigs. J Hyg (Lond) 1980; 85:275-85.
[24] Charland N, Jacques M, Lacouture S, Gottschalk M. Characterization and protective activity of a monoclonal antibody against a capsular epitope shared by Streptococcus suis serotypes 1, 2 and 1/2. Microbiology 1997; 143:3607-14.
[25] Knuf M, Kowalzik F, Kieninger D. Comparative effects of carrier proteins on vaccine-induced immune response. Vaccine 2011; 29:4881-90.
[26] Byrd W, Kadis S. Preparation, characterization, and immunogenicity of conjugate vaccines directed against Actinobacillus pleuropneumoniae virulence determinants. Infect Immun 1992; 60:3042-51.
[27] Byrd W, Harmon B G, Kadis S. Protective efficacy of conjugate vaccines against experimental challenge with porcine Actinobacillus pleuropneumoniae. Vet Immunol Immunopathol 1992; 34:307-24.
[28] Andresen L O, Jacobsen M J, Nielsen J P. Experimental vaccination of pigs with an Actinobacillus pleuropneumoniae serotype 5b capsular polysaccharide-tetanus toxoid conjugate. Acta Vet Scand 1997; 38:283-93.
[29] Van Calsteren M R, Gagnon F, Lacouture S, Fittipaldi N, Gottschalk M. Structure determination of Streptococcus suis serotype 2 capsular polysaccharide. Biochem Cell Biol 2010; 88:513-25.
[30] Segura M, Stankova J, Gottschalk M. Heat-killed Streptococcus suis capsular type 2 strains stimulate tumor necrosis factor alpha and interleukin-6 production by murine macrophages. Infect Immun 1999; 67:4646-54.
[31] Calzas C, Goyette-Desjardins G, Lemire P, Gagnon F, Lachance C, Van Calsteren M R, et al. Group B Streptococcus and Streptococcus suis capsular polysaccharides induce chemokine production by dendritic cells via Toll-like receptor 2- and MyD88-dependent and -independent pathways. Infect Immun 2013; 81:3106-18.
[32] Van Calsteren M R, Gagnon F, Calzas C, Goyette-Desjardins G, Okura M, Takamatsu D, et al. Structure determination of Streptococcus suis serotype 14 capsular polysaccharide. Biochem Cell Biol 2013; 91:49-58.
[33] Houde M, Gottschalk M, Gagnon F, Van Calsteren M-R, Segura M. Streptococcus suis Capsular Polysaccharide Inhibits Phagocytosis through Destabilization of Lipid Microdomains and Prevents Lactosylceramide-Dependent Recognition. Infect Immun 2012; 80:506-17.
[34] U.S. Code of Federal Regulations. Title 9-Animals and Animal Products, Chapter I-Animal and Plant Health Inspection Service, Department of Agriculture; Subchapter E; Part 117.4 Test animals. 1998.
[35] Higgins R, Gottschalk M. An update on Streptococcus suis identification. J Vet Diagn Invest 1990; 2:249-52.
[36] Goyette-Desjardins G, Roy R, Segura M. Murine whole-blood opsonophagocytosis assay to evaluate protection by antibodies raised against encapsulated extracellular bacteria. In: Lepenies B, editor. Methods in Molecular Biology—Carbohydrate-based vaccines. New York, USA: Springer; In press.
[37] Szu S C, Zon G, Schneerson R, Robbins J B. Ultrasonic irradiation of bacterial polysaccharides. Characterization of the depolymerized products and some applications of the process. Carbohydr Res 1986; 152:7-20.
[38] Reuter G, Schauer R, Szeiki C, Kamerling J P, Vliegenthart J F. A detailed study of the periodate oxidation of sialic acids in glycoproteins. Glycoconj J 1989; 6:35-44.
[39] Wessels M R, Paoletti L C, Kasper D L, DiFabio J L, Michon F, Holme K, et al. Immunogenicity in animals of a polysaccharide-protein conjugate vaccine against type III Group B Streptococcus. J Clin Invest 1990; 86:1428-33.
[40] Merril C, Goldman D, Sedman S, Ebert M. Ultrasensitive stain for proteins in polyacrylamide gels shows regional variation in cerebrospinal fluid proteins. Science 1981; 211:1437-8.
[41] Chu R S, Targoni O S, Krieg A M, Lehmann P V, Harding C V. CpG oligodeoxynucleotides act as adjuvants that switch on T helper 1 (Th1) immunity. J Exp Med 1997; 186:1623-31.
[42] Stills H F. Adjuvants and antibody production: dispelling the myths associated with Freund's complete and other adjuvants. ILAR Journal 2005; 46:280-93.
[43] Leenaars P P A M, Hendriksen C F M, Angulo A F, Koedam M A, Claassen E. Evaluation of several adjuvants as alternatives to the use of Freund's adjuvant in rabbits. Vet Immunol Immunopathol 1994; 40:225-41.
[44] Bennett B, Check I J, Olsen M R, Hunter R L. A comparison of commercially available adjuvants for use in research. J Immunol Methods 1992; 153:31-40.
[45] Sommariva M, de Cesare M, Meini A, Cataldo A, Zaffaroni N, Tagliabue E, et al. High efficacy of CpG-ODN, cetuximab and cisplatin combination for very advanced ovarian xenograft tumors. J Transl Med 2013; 11:25.
[46] Jennings V M. Review of selected adjuvants used in antibody production. ILAR Journal 1995; 37:119-25.
[47] Kateregga J, Lubega G W, Lindblad E B, Authie E, Coetzer T H, Boulange A F. Effect of adjuvants on the humoral immune response to congopain in mice and cattle. BMC Vet Res 2012; 8:63.
[48] Dalsgaard K, Hilgers L, Trouve G. Classical and new approaches to adjuvant use in domestic food animals. Adv Vet Sci Comp Med 1990; 35:121-60.
[49] Plotkin S A. Correlates of protection induced by vaccination. Clin Vaccine Immunol 2010; 17:1055-65.
[50] Swildens B, Nielen M, Wisselink H J, Verheijden J H M, Stegeman J A. Elimination of strains of Streptococcus suis serotype 2 from the tonsils of carrier sows by combined medication and vaccination. Vet Rec 2007; 160:619-21.
[51] Gilbert F B, Poutrel B, Sutra L. Immunogenicity in cows of Staphylococcus aureus type 5 capsular polysaccharide-ovalbumin conjugate. Vaccine 1994; 12:369-74.
[52] Lee C-J, Lee L H, Frasch C E. Protective immunity of pneumococcal glycoconjugates. Crit Rev Microbiol 2003; 29:333-49.
[53] Baker C J, Rench M A, Edwards M S, Carpenter R J, Hays B M, Kasper D L. Immunization of pregnant women with a polysaccharide vaccine of Group B Streptococcus. N Engl J Med 1988; 319:1180-5.
[54] Kasper D L, Paoletti L C, Wessels M R, Guttormsen H K, Carey V J, Jennings H J, et al. Immune response to type III Group B streptococcal polysaccharide-tetanus toxoid conjugate vaccine. J Clin Invest 1996; 98:2308-14.
[55] Moens L, Jeurissen A, Nierkens S, Boon L, Van Kaer L, Kasran A, et al. Generation of antibody responses to pneumococcal capsular polysaccharides is independent of CD1 expression in mice. Infect Immun 2009; 77:1976-80.
[56] Schütz K, Hughes R, Parker A, Quinti I, Thon V, Cavaliere M, et al. Kinetics of IgM and IgA antibody response to 23-valent pneumococcal polysaccharide vaccination in healthy subjects. J Clin Immunol 2013; 33:288-96.
[57] Baums C G, Kock C, Beineke A, Bennecke K, Goethe R, Schröder C, et al. Streptococcus suis bacterin and subunit vaccine immunogenicities and protective efficacies against serotypes 2 and 9. Clin Vaccine Immunol 2009; 16:200-8.
[58] Duan J, Kasper D L. Oxidative depolymerization of polysaccharides by reactive oxygen/nitrogen species. Glycobiology 2011; 21:401-9.
[59] Higashi K, Ly M, Wang Z, Masuko S, Bhaskar U, Sterner E, et al. Controlled photochemical depolymerization of K5 heparosan, a bioengineered heparin precursor. Carbohydr Polym 2011; 86:1365-70.
[60] Anderson P. Antibody responses to Haemophilus influenzae type b and diphtheria toxin induced by conjugates of oligosaccharides of the type b capsule with the nontoxic protein CRM197. Infect Immun 1983; 39:233-8.
[61] Svenson S B, Lindberg A A. Oligosaccharide-protein conjugate: A novel approach for making Salmonella 0-antigen immunogens. FEMS Microbiol Lett 1977; 1:145-7.
[62] Svenson S B, Lindberg A A. Coupling of acid labile Salmonella specific oligosaccharides to macromolecular carriers. J Immunol Methods 1979; 25:323-35.
[63] Wessels M R, Muńoz A, Kasper D L. A model of high-affinity antibody binding to type III Group B Streptococcus capsular polysaccharide. Proc Natl Acad Sci 1987; 84:9170-4.
[64] Paoletti L C, Kasper D L, Michon F, DiFabio J, Holme K, Jennings H J, et al. An oligosaccharide-tetanus toxoid conjugate vaccine against type III Group B Streptococcus. J Biol Chem 1990; 265:18278-83.
[65] Pawlowski A, Svenson S B. Electron beam fragmentation of bacterial polysaccharides as a method of producing oligosaccharides for the preparation of conjugate vaccines. FEMS Microbiol Lett 1999; 174:255-63.
[66] Paoletti L C, Wessels M R, Michon F, DiFabio J, Jennings H J, Kasper D L. Group B Streptococcus type II polysaccharide-tetanus toxoid conjugate vaccine. Infect Immun 1992; 60:4009-14.
[67] Wessels M R, Paoletti L C, Rodewald A K, Michon F, DiFabio J, Jennings H J, et al.
Stimulation of protective antibodies against type Ia and Ib Group B Streptococci by a type Ia polysaccharide-tetanus toxoid conjugate vaccine. Infect Immun 1993; 61:4760-6.
[68] Paoletti L C, Wessels M R, Rodewald A K, Shroff A A, Jennings H J, Kasper D L. Neonatal mouse protection against infection with multiple Group B streptococcal (GBS) serotypes by maternal immunization with a tetravalent GBS polysaccharide-tetanus toxoid conjugate vaccine. Infect Immun 1994; 62:3236-43.
[69] Wessels M R, Paoletti L C, Pinel J, Kasper D L. Immunogenicity and protective activity in animals of a type V Group B streptococcal polysaccharide-tetanus toxoid conjugate vaccine. J Infect Dis 1995; 171:879-84.
[70] Paoletti L C, Pinel J, Johnson K D, Reinap B, Ross R A, Kasper D L. Synthesis and preclinical evaluation of glycoconjugate vaccines against Group B Streptococcus types VI and VIII. J Infect Dis 1999; 180:892-5.
[71] Paoletti L C, Kasper D L. Conjugate vaccines against Group B Streptococcus types IV and VII. J Infect Dis 2002; 186:123-6.
[72] Wessels M R, Paoletti L C, Guttormsen H K, Michon F, D'Ambra A J, Kasper D L. Structural properties of Group B streptococcal type III polysaccharide conjugate vaccines that influence immunogenicity and efficacy. Infect Immun 1998; 66:2186-92.
[73] Jennings H J, Lugowski C, Kasper D L. Conformational aspects critical to the immunospecificity of the type III Group B streptococcal polysaccharide. Biochemistry 1981; 20:4511-8.
[74] Zou W, Mackenzie R, Thérien L, Hirama T, Yang Q, Gidney M A, et al. Conformational epitope of the type III Group B Streptococcus capsular polysaccharide. J Immunol 1999; 163:820-5.
[75] Avci F Y, Li X, Tsuji M, Kasper D L. A mechanism for glycoconjugate vaccine activation of the adaptive immune system and its implications for vaccine design. Nat Med 2011; 17:1602-9.
[76] Michon F, Uitz C, Sarkar A, D'Ambra A J, Laude-Sharp M, Moore S, et al. Group B streptococcal type II and III conjugate vaccines: physicochemical properties that influence immunogenicity. Clin Vaccine Immunol 2006; 13:936-43.
[77] O'Hagan D T, Valiante N M. Recent advances in the discovery and delivery of vaccine adjuvants. Nat Rev Drug Discov 2003; 2:727-35.
[78] Chu R S, McCool T, Greenspan N S, Schreiber J R, Harding C V. CpG oligodeoxynucleotides act as adjuvants for pneumococcal polysaccharide-protein conjugate vaccines and enhance antipolysaccharide immunoglobulin G2a (IgG2a) and IgG3 antibodies. Infect Immun 2000; 68:1450-6.
[79] Fattom A, Li X, Cho Y H, Burns A, Hawwari A, Shepherd S E, et al. Effect of conjugation methodology, carrier protein, and adjuvants on the immune response to Staphylococcus aureus capsular polysaccharides. Vaccine 1995; 13:1288-93.
[80] Bode C, Zhao G, Steinhagen F, Kinjo T, Klinman D M. CpG DNA as a vaccine adjuvant. Expert Rev Vaccines 2011; 10:499-511.
[81] Kovarik J, Bozzotti P, Tougne C, Davis H L, Lambert P H, Krieg A M, et al. Adjuvant effects of CpG oligodeoxynucleotides on responses against T-independent type 2 antigens. Immunology 2001; 102:67-76.
[82] Cox J C, Coulter A R. Adjuvants—A classification and review of their modes of action. Vaccine 1997; 15:248-56.
[83] Leenaars P P, Savelkoul H F, Hendriksen C F, Van Rooijen N, Claassen E. Increased adjuvant efficacy in stimulation of antibody responses after macrophage elimination in vivo. Immunology 1997; 90:337-43.
[84] Heegaard P, Dedieu L, Johnson N, Le Potier M-F, Mockey M, Mutinelli F, et al. Adjuvants and delivery systems in veterinary vaccinology: current state and future developments. Arch Virol 2011; 156:183-202.
[85] Hunter R L. Overview of vaccine adjuvants: present and future. Vaccine 2002; 20 Suppl 3:S7-12.
[86] Peeters C C, Lagerman P R, Weers O, Oomen L A, Hoogerhout P, Beurret M, et al. Preparation of polysaccharide-conjugate vaccines. In: Robinson A, Hudson M J, Cranage M P, editors. Methods in Molecular Medecine—Vaccine Protocols. 2nd ed. Totowa, USA: Humana Press Inc; 2003. p. 153-73.
[87] Wilson D, Braley-Mullen H. Antigen requirements for priming of type III pneumococcal polysaccharide-specific IgG memory responses: suppression of memory with the T-independent form of antigen. Cell Immunol 1981; 64:177-86.
[88] Peeters CCAM, Tenbergen-Meekes A-M J, Poolman J T, Zegers B J M, Rijkers G T. Immunogenicity of a Streptococcus pneumoniae type 4 polysaccharide-protein conjugate vaccine is decreased by admixture of high doses of free saccharide. Vaccine 1992; 10:833-40.
[89] Rodriguez M E, van den Dobbelsteen G P, Oomen L A, de Weers O, van Buren L, Beurret M, et al. Immunogenicity of Streptococcus pneumoniae type 6B and 14 polysaccharide-tetanus toxoid conjugates and the effect of uncoupled polysaccharide on the antigen-specific immune response. Vaccine 1998; 16:1941-9.
[90] Romero-Steiner S, Frasch C E, Carlone G, Fleck R A, Goldblatt D, Nahm M H. Use of opsonophagocytosis for serological evaluation of pneumococcal vaccines. Clin Vaccine Immunol 2006; 13:165-9.
[91] Guilliams M, Bruhns P, Saeys Y, Hammad H, Lambrecht B N. The function of Fcg receptors in dendritic cells and macrophages. Nat Rev Immunol 2014; 14:94-108.
[92] Underhill D M, Ozinsky A. Phagocytosis of microbes: complexity in action. Annu Rev Immunol 2002; 20:825-52.
[93] Ricklin D, Hajishengallis G, Yang K, Lambris J D. Complement: a key system for immune surveillance and homeostasis. Nat Immunol 2010; 11:785-97.
[94] Perlmutter R M, Hansburg D, Briles D E, Nicolotti R A, David J M. Subclass Restriction of Murine Anti-Carbohydrate Antibodies. J Immunol 1978; 121:566-72.
[95] Rubinstein L J, Stein K E. Murine immune response to the Neisseria meningitidis group C capsular polysaccharide. I. Ontogeny. J Immunol 1988; 141:4352-6.
[96] Schreiber J R, Cooper L J N, Diehn S, Dahlhauser P A, Tosi M F, Glass D D, et al. Variable region-identical monoclonal antibodies of different IgG subclass directed to Pseudomonas aeruginosa lipopolysaccharide 0-specific side chain function differently. J Infect Dis 1993; 167:221-6.
[97] Michaelsen T E, Kolberg J, Aase A, Herstad T K, Høiby E A. The four mouse IgG isotypes differ extensively in bactericidal and opsonophagocytic activity when reacting with the P1.16 epitope on the outer membrane PorA protein of Neisseria meningitidis. Scand J Immunol 2004; 59:34-9.
[98] McLay J, Leonard E, Petersen S, Shapiro D, Greenspan N S, Schreiber J R. γ3 gene-disrupted mice selectively deficient in the dominant IgG subclass made to bacterial polysaccharides. I I. Increased susceptibility to fatal pneumococcal sepsis due to absence of anti-polysaccharide IgG3 is corrected by induction of anti-polysaccharide IgG1. J Immunol 2002; 168:3437-43.
[99] Galina L, Vecht U, Wisselink H J, Pijoan C. Prevalence of various phenotypes of Streptococcus suis isolated from swine in the U.S.A. based on the presence of muraminidase-released protein and extracellular factor. Can J Vet Res 1996; 60:72-4.
Gottschalk M, Lebrun A, Wisselink H, Dubreuil J D, Smith H, Vecht U. Production of virulence-related proteins by Canadian strains of Streptococcus suis capsular type 2. Can J Vet Res 1998; 62:75-9.
Okwumabua O, Abdelmagid O, Chengappa M M. Hybridization analysis of the gene encoding a hemolysin (suilysin) of Streptococcus suis type 2: evidence for the absence of the gene in some isolates. FEMS Microbiol Lett 1999; 181:113-21.
Fittipaldi N, Xu J, Lacouture S, Tharavichitkul P, Osaki M, Sekizaki T, et al. Lineage and virulence of Streptococcus suis serotype 2 isolates from North America. Emerg Infect Dis 2011; 17:2239-44.
Li Y, Martinez G, Gottschalk M, Lacouture S, Willson P, Dubreuil J D, et al. Identification of a surface protein of Streptococcus suis and evaluation of its immunogenic and protective capacity in pigs. Infect Immun 2006; 74:305-12.

What is claimed is:

1. An immunogenic composition, comprising: a capsular polysaccharide-protein conjugate, together with a physiologically acceptable vehicle, wherein said conjugate comprises a capsular polysaccharide from Streptococcus suis conjugated to a carrier protein, wherein in said capsular polysaccharides are prepared from Streptococcus suis serotypes 1, 2, 7 or 9, or any other serotype or combinations thereof.

2. The immunogenic composition of claim 1, wherein said carrier protein is selected from the group comprising inactivated bacterial toxins, bacterial outer membrane proteins, ovalbumin, keyhole limpet hemocyanin (KLH), bovine serum albumin (BSA), or tuberculin.

3. The immunogenic composition of claim 1, wherein said carrier protein is an inactivated bacterial toxin selected of the group comprising tetanus toxoid, diphtheria toxoid, non-toxic cross-reactive material of diphtheria toxin (CRM197), pertussis toxoid, cholera toxoid, E. coli LT, E. coli ST, and exotoxin A from Pseudomona aeruginosa, or any other typical protein carrier used in humans, or any immunogenic peptide/fragments derived from the above.

4. The immunogenic composition of claim 3, wherein said carrier protein is tetanus toxoid.

5. The immunogenic composition of claim 1, wherein said carrier protein is selected from the group comprising S. suis-derived immunogenic somatic and/or secreted protein selected from, but not restricted to, the group comprising suilysin, MRP, EF, enolase, subtilisin, and DNAse.

6. The immunogenic composition of claim 1, wherein said capsular polysaccharide from S. suis is from serotype 2.

7. The immunogenic composition of claim 1, wherein said capsular polysaccharide from S. suis is from serotype 1.

8. The immunogenic composition of claim 1, wherein said capsular polysaccharide from S. suis is from serotype 7.

9. The immunogenic composition of claim 1, wherein said capsular polysaccharide from S. suis is from serotype 9.

10. The immunogenic composition of claim 5, wherein said polysaccharides are conjugated to the carrier protein tetanus toxoid.

11. A multivalent immunogenic composition, comprising: polysaccharide-protein conjugates prepared from at least two different S. suis serotypes, together with a physiologically acceptable vehicle, wherein each conjugate comprises a capsular polysaccharide from Streptococcus suis conjugated to a carrier protein, wherein in said capsular polysaccharides are prepared from Streptococcus suis serotypes 1, 2, 7 and/or 9 or any other serotype.

12. The multivalent immunogenic composition of claim 10, wherein said carrier protein is selected from the group comprising inactivated bacterial toxins, bacterial outer membrane proteins, ovalbumin, keyhole limpet hemocyanin (KLH), bovine serum albumin (BSA), or tuberculin.

13. The multivalent immunogenic composition of claim 12, wherein said carrier protein is selected from the group comprising tetanus toxoid, diphtheria toxoid, non-toxic cross-reactive material of diphtheria toxin (CRM197), pertussis toxoid, cholera toxoid, E. coli LT, E. coli ST, and exotoxin A from Pseudomona aeruginosa, or any other typical protein carrier used in humans, or any immunogenic peptide/fragments derived from the above

14. The immunogenic composition of claim 12, wherein said carrier protein is selected from the group comprising S. suis-derived immunogenic somatic and/or secreted protein selected from, but not restricted to, the group comprising suilysin, MRP, EF, enolase, subtilisin, and DNAse.

15. The multivalent immunogenic composition of claim 12, wherein each capsular polysaccharide is separately conjugated to tetanus toxoid carrier protein.

16. A method of reducing S. suis associated impaired behavior, lameness, frequency of brain lesions and central nervous system-associated clinical signs, bacteremia, recovery and/or colonization of bacterium from internal tissues, inflammation in thoracic and abdominal cavities, and mortality in swine comprising the administration of the immunogenic composition of claim 1 to an animal in need thereof.

17. The method of claim 16, comprising the administration of the immunogenic composition of claim 6 to an animal in need thereof.

18. The method of claim 16, comprising the administration of the immunogenic composition of claim 7 to an animal in need thereof.

19. The method of claim 16, comprising the administration of the immunogenic composition of claim 8 to an animal in need thereof.

20. The method of claim 16, comprising the administration of the immunogenic composition of claim 9 to an animal in need thereof.
